(19) United States
(12) Patent Application Publication
(10) Pub. No.: US 2015/0073324 A1
(43) Pub. Date: Mar. 12, 2015

(54) CELL FORMING STRUCTURES

(71) Applicant: The Procter & Gamble Company, Cincinnati, OH (US)

(72) Inventors: Tina Liebe, Schwalbach (DE); Matthias Konrad Hippe, Sulzbach (DE)

(21) Appl. No.: 14/479,783

(22) Filed: Sep. 8, 2014

Related U.S. Application Data
(63) Continuation of application No. 14/022,266, filed on Sep. 10, 2013.

(51) Int. Cl. B32B 3/16; B65D 81/03; A61F 13/00

(52) U.S. Cl. CPC B32B 3/16; A61F 13/00051; B65D 81/03; A61F 2013/00272; B32B 2553/02; B32B 2250/03
USPC 602/44; 602/45; 602/46; 602/43; 428/101; 428/34.1

(57) ABSTRACT

The invention refers to a disposable absorbent article such as a diaper, a pant or a sanitary napkin. The disposable absorbent article further comprises a structure which is able to elongate and simultaneously convert from an initial flat configuration into an erected configuration.

### BACKGROUND OF THE INVENTION

The use of soft, flexible and extensible materials as well as their use in a large variety of consumer products is well known in the an. Often, it is also desirable that these materials exhibit elastic-like behavior, such that they return to essentially their initial form when an applied force, by which the material has been converted into an elongated state, is removed.

A drawback commonly associated with extensible materials and elastic materials, such as (elastic) films or nonwoven webs, is that their width decreases when they are elongated along their lengthwise dimension. This property is typically referred to as necking. Also, extensible as well as elastic materials typically decrease in caliper, i.e. in thickness, when being elongated.

Generally, materials which increase in thickness when being stretched are also known in the art. These so-called “auxetics” are materials which have a negative Poisson's ratio. When stretched, they become thicker perpendicular to the applied force. This behavior is due to their hinge-like structures, which flex when stretched. Auxetic materials can be single molecules or a particular structure of macroscopic matter. Such materials are expected to have mechanical properties such as high energy absorption and fracture resistance. Auxetics have been described as being useful in applications such as body armor, packing material, knee and elbow pads, robust shock absorption material, and sponge mops. Typically, though their thickness increases upon elongation, (macroscopic) auxetic materials have a relatively significant thickness already in their relaxed state. That is, known auxetic structures are typically non-flat structures having predominantly 3-dimensional shape when they are in their relaxed state.

There is still a need for extensible structures and which increase in caliper when being stretched. Further, it would be desirable that these structures show auxetic behavior in that they increase in caliper (i.e. thickness) upon being stretched, while the structures should desirably be relatively flat in their initial, non-stretched state.

Such structures may also exhibit elastic-like behavior such that they can retract to substantially their initial shape when an applied force, upon which the structure is converted them into an elongated shape with increased caliper, is removed. Alternatively, the structures may be facilitated such that the structure, once elongated, tends to remain substantially in its elongated configuration with increased caliper when the applied force is removed. Also, structures may convert to an intermediate configuration when the applied force is removed.

It would also be desirable to be able to make such structures from relatively inexpensive, widely available feedstock materials.

Such structures would have wide applicability, for example in disposable consumer products, such as wound dressings. Especially, for products winch are typically densely packed while the product is in a flat configuration, the use of such structures would be attractive.

### SUMMARY OF THE INVENTION

The invention refers to a structure having a longitudinal dimension and a lateral dimension perpendicular to the longitudinal dimension. Upon application of a force along the longitudinal dimension of the structure, the structure is able to elongate along the longitudinal dimension, whereby the structure is simultaneously able to convert from an initial flat configuration into an erected configuration.

The structure comprises a first and a second layer. Each layer has an inner and an outer surface, a longitudinal dimension parallel to the longitudinal dimension of the structure confined by two spaced apart lateral edges, and a lateral dimension parallel to the lateral dimension of the structure confined by two spaced apart longitudinal edges.

The first and second layer at least partly overlap each other, wherein the first layer is able to shift relative to the second layer in opposite directions along the longitudinal structure dimension upon application of a force along the longitudinal dimension.

The structure farther comprises ligaments provided between the first and second layer in at least a part of the region where the first and second layer overlap each other. Each ligament has a longitudinal dimension confined by two spaced apart lateral ligament edges, and a lateral dimension confined by two spaced apart longitudinal ligament edges.

The first and second layers and the ligaments of the structure are made of film, nonwoven material, paper, sheet-like foam, woven fabric, knitted fabric or combinations thereof.

Each ligament is attached to the inner surface of the first layer with a portion at or adjacent to one of the ligament's lateral edges in a first ligament attachment region, and is further attached to the inner surface of the second layer with a portion at or adjacent to the ligament's other lateral edge in a second ligament attachment region. The region between the first and second ligament attachment region forms a free intermediate portion.

The ligaments are spaced apart from one another along the longitudinal dimension of the structure, and the attachment of all ligaments to the first and second layer in the first and second ligament attachment regions is such that the free intermediate portions of the ligaments are able to convert from an initial flat configuration to an erected configuration upon application of a force along the longitudinal dimension of the structure, thus converting the structure as a whole from an initial flat configuration into an erected configuration, with the erection being in a direction perpendicular to the longitudinal and the lateral dimension of the structure (i.e. the caliper of the structure increases compared to the initial flat configuration).

For all ligaments in the structure, a first surface of the free intermediate portion may face towards the inner surface of the first layer and a second surface of the free intermediate portion may face towards the inner surface second layer in the structure's flat configuration, and the first surface of the free intermediate portion may face towards the second surface of the neighboring ligament when the structure is in the erected configuration.

For all ligaments in a structure, the ligament's first surface may not be facing towards the inner surface of the second layer when the structure is in its initial flat configuration, and the ligament's second surface may not be facing towards the inner surface of the first layer when the structure is in its initial flat configuration.

When the ligaments are in their erected configuration, each ligament may either adopt a Z-like shape, a C-like shape, a shape forming a T in one of the first or second ligament attachment region and taking a tilted shape in the other of the first or second ligament attachment region (hereinafter simply referred to as “T-like shape”), or a Double-T-like shape.

All ligaments in a structure adopting a Z-like shape may adopt a Z-like shape with the same orientation. Likewise, all ligaments in a structure adopting a C-like shape may adopt a C-like shape with the same orientation when the structure is converted into its erected configuration. Hence, no ligaments in a structure adopting a Z-like shape or a C-like shape may have an inverted configuration versus another ligament in the structure with a Z-like or C-like shape.

The names “Z-like shape”, “C-like shape”, “T-like shape”, and “Double-T-like shape” reflect the appearance and shape of the ligaments when viewed from the side parallel to the lateral direction and seen between the first and second layer.

All, ligaments in a structure, which may adopt a Z-like shape, may adopt a Z-like shape with the same orientation and/or all ligaments in a structure, which may adopt a C-like shape, may adopt a C-like shape with the same orientation when the structure is converted into its erected configuration.

Also, structures are provided wherein all ligaments may adopt a Z-like shape with the same orientation when the structure is converted into its erected configuration.

The structure may comprise one or more stop aid(s) which define(s) the maximum shifting of the first layer relative to the second layer along the longitudinal dimension in opposite directions when the force along the longitudinal dimension is applied, wherein the maximum shifting defined by the stop aid is less than the maximum shifting provided by the ligaments in the absence of such stop aid.

In the absence of a stop aid, which may be comprised by the structure or which may be external to the structure but having the same effect as a stop aid comprised by the structure, the first layer would be able to continue shifting relative to the second layer in opposite directions along the longitudinal structure dimension upon continued application of a force along the longitudinal dimension structure when the structure is in its erected configuration. Thereby, the ligaments' free intermediate portion would turn over up to about 180° from their position in the initial flat structure configuration to an erected configuration and into a turned-over flat structure configuration. In its final, turned-over flat structure configuration, it would not be possible to elongate the structure any further along the longitudinal dimension, unless at least the first and second layer—and possibly also the ligaments—are made of extensible or elastic material.

In the structure, the free intermediate portion of each ligament may either remain unattached to the first and second layer or may be releasable attached to the first layer, to the second layer or to the first and second layer. In the structure, the free intermediate portions of the ligaments may not be attached to each other. Alternatively, in the structure, the free intermediate portions of neighboring ligaments may be attached to each other either directly, or indirectly via an additional piece of material, wherein the attachment may be either permanent or releasable.

In the structure, the tree intermediate portion of each ligament may have the same longitudinal dimension. Alternatively, in the structure, the longitudinal dimension of the free intermediate portion of one or more of the ligaments may differ from the longitudinal dimension of the free intermediate portion of one or more other ligaments.

The ligaments comprised by the structure may be spaced apart from each other at equal distances. Alternatively, the ligaments may be spaced apart from each other at varying distances.

The ligaments comprised by the structure may be spaced apart from each other such that the ligaments do not overlap with each other when the structure is in its initial flat configuration.

The ligaments of the structure may have a tensile strength of from about 1 N/cm to about 100N/cm and/or may have a bending stiffness of from about 0.01 mNm to about 1 Nm.

The ligaments of the structure may differ from each other in tensile strength, in bending stiffness or in tensile strength and bending stiffness.

### BRIEF DESCRIPTION OF THE DRAWINGS

These and other features, aspects and advantages of the present invention will become better understood with regard to the following description, appended claims, and accompanying drawing where:

FIG. 1A is a side view (parallel to the lateral dimension) of an embodiment of the structure of the present invention—with ligaments having Z-like shape, wherein the structure is in its initial flat configuration and wherein the free intermediate portion of all ligaments has the same length.

FIG. 1B is a side view of the embodiment of FIG. 1A, wherein the structure is now in its erected configuration

FIG. 2 is a side view (parallel to the lateral dimension) of an embodiment of the structure of the present invention—with ligaments having Z-like shape—, wherein the structure is in its erected configuration and wherein the free intermediate portion of the ligaments varies with the central ligament having the biggest length

FIG. 3 is a side view (parallel to the lateral dimension) of an embodiment of the structure of the present invention—with ligaments having Z-like shape—wherein the structure is in its erected configuration and wherein the free intermediate portion of the ligaments varies with the ligament towards one lateral edge of the structure having the biggest length

FIG. 4A is a side view (parallel to the lateral dimension) of an embodiment of the structure of the present invention—with ligaments having Z-like shape, wherein the structure is in its initial flat configuration and wherein the structure comprises a layer-to-layer stop aid

FIG. 4B is a side view (parallel to the lateral dimension) of the embodiment of FIG. 4A, now in its erected configuration.

FIG. 5A is a side view (parallel to the lateral dimension) of an embodiment of the structure of the present invention—with ligaments having Z-like shape, wherein the structure is in its initial flat configuration and wherein the structure comprises a layer-to-ligament stop aid

FIG. 5B is a side view (parallel to the lateral dimension) of the embodiment of FIG. 5A, now in its erected configuration

FIG. 6 is a side view of the structure shown in FIG. 1B which comprises a ligament-to-ligament material between neighboring ligaments

FIG. 7 is a side view of another embodiment of the structure of the present invention—with ligaments having Z-like shape

FIG. 8A is a side view of an embodiment of the structure of the present invention—with ligaments having C-like shape, wherein the structure is in its initial flat configuration

FIG. 8B is a side view of the embodiment of FIG. 8A, wherein the structure is in its partly erected configuration

FIG. 8C is a side view of the embodiment of FIG. 8A, wherein the structure is in its maximum erected configuration

FIG. 9A is a side view of an embodiment of the structure of the present invention—with ligaments having Double-T-like shape—, wherein the structure is in its initial flat configuration

FIG. 9B is a side view of the embodiment of FIG. 9A, wherein the structure is in its partly erected configuration

FIG. 9C is a side view of the embodiment of FIG. 9A, wherein the structure is in its maximum erected configuration

FIG. 10A is a side view of an embodiment of the structure of the present invention—with ligaments having T-like shape—, wherein the structure is in its initial flat configuration

FIG. 10B is a side view of the embodiment of FIG. 10A, wherein the structure is in its partly erected configuration

FIG. 10C is a side view of the embodiment of FIG. 10A, wherein the structure is in its maximum erected configuration

FIG. 11A is a side view of an embodiment of the structure of the present invention—with ligaments of Z-like shape having inverse configuration, wherein the structure is in its initial flat configuration

FIG. 11B is a side view of the embodiment of FIG. 11A, wherein the structure is in its erected configuration

FIG. 12A is a side view of an embodiment of the structure of the present invention—with ligaments of C-like shape having inverse configuration, wherein the structure is in its initial flat configuration

FIG. 12B is a side view of the embodiment of FIG. 12A, wherein the structure is in its erected configuration

FIG. 13 is a side view of an embodiment of the structure not comprised by the present invention, wherein the structure cannot be converted into an erected configuration

FIG. 14A is a side view of an embodiment of the structure of the present invention—with ligaments made of two-layered laminates and having Double-T-like shape—, wherein the structure is in its initial flat configuration

FIG. 14B is a side view of the embodiment of FIG. 14A, wherein the structure is in its partly erected configuration

FIG. 14C is a side view of the embodiment of FIG. 14A, wherein the structure is in its maximum erected configuration

FIG. 15A is a side view of another embodiment of the structure of the present invention—with ligaments compiled of separate pieces of material and having Double-T-like shape—, wherein the structure is in its initial flat configuration

FIG. 15B is a side view of the embodiment of FIG. 15A, wherein the structure is in its partly erected configuration

FIG. 15C is a side view of the embodiment of FIG. 15A, wherein the structure is in its maximum erected configuration

FIG. 16A is a side view of another embodiment of the structure of the present invention—with ligaments compiled of separate pieces of material and having Z-like shape—, wherein the structure is in its initial flat configuration

FIG. 16B is a side view of the embodiment of FIG. 16A, wherein the structure is in its partly erected configuration

FIG. 16C is a side view of the embodiment of FIG. 16A, wherein the structure is in its maximum erected configuration

FIG. 17A is a side view of another embodiment of the structure of the present invention—with ligaments having cut out areas and having Z-like shape—, wherein the structure is substantially in its initial flat configuration

FIG. 17B is a side view of the embodiment of FIG. 17A, wherein the structure is in its erected configuration

FIG. 18 is a schematic drawing of parts of the equipment used for the modulus test method

### DETAILED DESCRIPTION OF THE INVENTION

### Definitions

“Bandage” as used herein, refers to a A bandage is a piece of material used either to support a medical device such as wound dressing, or on its own to provide support to the body. Bandages may be used, e.g. during heavy bleeding or following a poisonous bite, in order to slow the flow of blood. Bandages are available in a wide range of types, from generic cloth strips to specialized shaped bandages designed for a specific limb or part of the body. While a wound dressing is in direct contact with a wound, a bandage is not directly in contact with a wound but may be used to support a wound dressing.

“Consumer product” as used herein, refers to an article produced or distributed for sale to a consumer for the personal use, consumption or enjoyment by a consumer in or around a permanent or temporary household or residence. A consumer product is not used in the production of another good.

“Disposable” is used in its ordinary sense to mean product that is disposed or discarded after a limited number of usage over varying lengths of time, for example, less than 20 usages, less than 10 usages, less than 5 usages, or after single use.

The term “film” as used herein refers to a substantially non-fibrous sheet-like material wherein the length and width of the material far exceed the thickness of the material. Typically, films have a thickness of about 0.5 mm or less, Films may be configured to be liquid impermeable and/or vapor permeable (i.e., breathable). Films may be made of polymeric, thermoplastic material, such as polyethylene, polypropylene or the like.

“Non-extensible” as used herein refers to a material which, upon application of a force, elongates beyond its original length by less than 20% if subjected to the following test:

A rectangular piece of the material having a width of 2.54 cm and a length of 25.4 cm is maintained in a vertical position by holding the piece along its upper 2.54 cm wide edge along its complete width. A force of 10 N is applied onto the opposite lower edge along the complete width of the material for 1 minute (at 25° C. and 50% rel. humidity; samples should be preconditioned at these temperature and humidity conditions for 2 hours prior to testing).

Immediately after one minute, the length of the piece is measured while the force is still applied and the degree of elongation is calculated by subtracting the initial length (25.4 cm) from the length measured after one minute.

If a material elongates beyond its original length by more than 20% if subjected to the above described test, it is “extensible” as used herein.

“Highly non-extensible” as used herein refers to a material, which, upon application of a force, elongates beyond its original length by less than 10% if subjected to the test described above for “non-extensible” material.

“Non-elastic” as used herein refers to a material which does not recover by more than 20% if subjected to the following test, which is to be carried out immediately subsequent to the test on “non-extensibility” set out above.

Immediately after the length of the rectangular piece of material has been measured while the ION force is still applied, the force is removed and the piece is laid down flat on a table for 5 minutes (at 25° C. and 50%) rel. humidity) to be able to recover. Immediately after 5 minutes, the length of the piece is measured again and the degree of elongation is calculated by subtracting the initial length (25.4 cm) from the length after 5 minutes.

The elongation after one minute while the force has been applied (as measured with respect to “non-extensibility”) is compared to the elongation after the piece has been laid down flat on a table for 5 minutes: If the elongation does not recover by more than 20%, the material is considered to be “non-elastic”.

If a material recovers by more than 20%, the material is considered “elastic” as used herein.

“Highly non-elastic” as used herein refers to a material, which is either “non-extensible” or which does not recover by more than 10% if subjected to the test set out above for “non-elastic”.

For use in the cell forming structures of the present invention, extensible, non-extensible, highly non-extensible, elastic, non-elastic and highly non-elastic relate to the dimension of the material, which, once the material has been incorporated into the structure, is parallel to the longitudinal dimension of the structure. Hence, the sample length of 25.4 cm for carrying out the tests described above corresponds to the longitudinal dimension of the cell forming structure once the material has been incorporated into the structure.

A “nonwoven web” is a manufactured web of directionally or randomly oriented fibers, consolidated and bonded together. The term does not include fabrics which are woven, knitted, or stitch-bonded with yarns or filaments. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. Nonwoven webs may be bonded by heat and/or pressure or may be adhesively bonded. Bonding may be limited to certain areas of the nonwoven web (point bonding, pattern bonding). Nonwoven webs may also be hydro-entangled or needle-punched. The basis weight of nonwoven fabrics is usually expressed in grams per square meter (g/m²).

A “paper” refers to a wet-formed fibrous structure comprising cellulose fibers,

“Sheet-like foam”, as used herein is a solid sheet that is formed by trapping pockets of gas. The solid foam may be closed-cell foam or open-cell foam. In closed-cell foam, the gas forms discrete pockets, each completely surrounded by the solid material. In open-cell foam, the gas pockets connect with each other.

“Sheet-like” means that the length and width of the material far exceed the thickness of the material.

“Wound dressing”, as used herein, is used to cover and protect a wound in order to promote healing and/or prevent further harm.

### Cell Forming Structures

For many applications, such as many applications in disposable consumer products, it would be highly desirable to have structures, which are initially flat but which simultaneously increase in caliper (i.e. thickness) when being elongated along their longitudinal dimension.

Moreover, such structures may exhibit an elastic-like behavior, i.e. they are able return—at least to some extent—to their initial longitudinal dimension and also to their initial caliper. Alternatively, the structure may be facilitated such that, once elongated, it remains substantially in its elongated configuration with increased caliper when the applied force is removed. In a still further alternative, structures may convert to an intermediate configuration with a length and caliper in between the initial state and their stretched state when the applied force is removed.

The present invention relates to so-called cell forming structures (herein referred to simply as “structures”) due to the cells formed between neighboring ligaments in the erected structure configuration, the cells being delimited by two neighboring ligaments and the first and second layer. These structures are initially relatively flat. When a force is applied along the longitudinal dimension (i.e. along the lengthwise extension) of the structure, the structure elongates and simultaneously adopts an erected configuration. Thus, the structure increases in caliper. As used herein, the terms “caliper” and “thickness” are used interchangeably and refer to a direction perpendicular to the lateral and longitudinal dimension. Moreover, when the applied force is released, these structures may be able to revert, to substantially their initial flat and shortened configuration. Such structures can be elongated and relaxed repeatedly. It is also possible to put the structure into execution such that the elongated structure does not or only to a certain extent return to its initial flat and shortened configuration when the applied force is released.

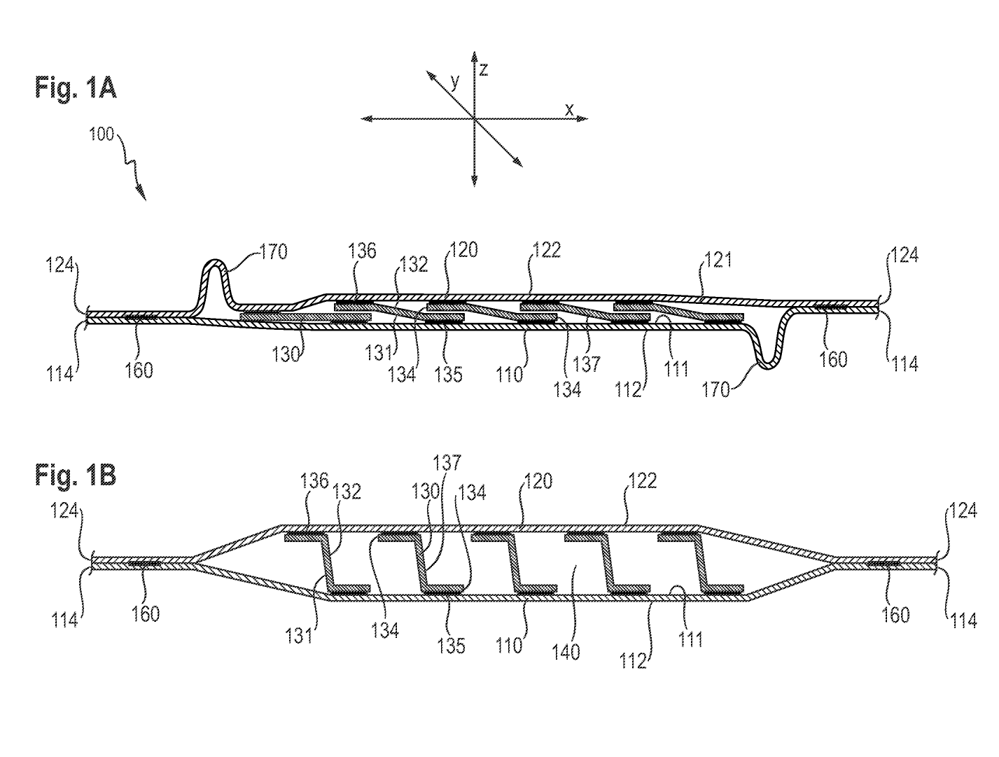

FIG. 1A shows a cell-forming structure—with ligaments having Z-like shape—in its flat configuration whereas FIG. 1B shows the structure in its erected configuration.

Generally, the structure (100) of the present invention comprises a first and a second layer (110, 120). One or both of the first and second layers (110, 120) may be non-elastic or highly non-elastic. Also one or both of the first and second layers may be non-extensible or highly non-extensible. Given that elastic materials are often more expensive compared to non-elastic materials, it may be advantageous to use non-elastic, or highly non-elastic materials for the first and second layer.

Moreover, if the first and second layers are non-elastic, the overall structure may be more easily and reliably transferred from its initial flat configuration into its erected configuration, as the applied force is more readily used to erect the structure. If the first and second layers are elastic, the applied forces may partly be converted into elongation of the first and second layer alone, i.e. they are not used to erect the structure as a whole, depending on the elastic modulus of the elastic material. However, the use of elastic materials or highly elastic materials for the first and second layer is also possible, especially if the elastic modulus is selected appropriately (typically, the elastic modulus should be relatively high). Similar considerations principally also apply to the use of extensible or highly extensible materials for the first and second layer.

The first and second layers are made of nonwovens, film, paper, sheet-like foam, woven fabric, knitted fabric or combinations of these materials. Combinations of these materials may be laminates, e.g. a laminate of a film and a nonwoven. Generally, a laminate may consist of only two materials joined to each other in a face to face relationship and lying upon another but alternatively may also comprise more than two materials joined to each other in a face to face and lying upon another.

The first and second layer may be made of the same material. Alternatively, the first layer may be made of material which is different from the material of the second layer.

The first layer and the second layer may also be made of a single, continuous material which is folded over at one of the lateral edges of the structure. In such embodiments, one of the lateral edges of the first layer is coincident with one of the lateral edges of the second layer, as these lateral edges are located at the interface of the first and second layer. However, alternatively, the first and second layer may be made of two separate pieces of material.

The materials of the first and second layer may be chosen such that the first and second layers have the same basis weight, tensile strength, bending stiffness, liquid permeability, breathability and/or hydrophilicity. Alternatively, the first and second layer may differ from each other in one or more properties, such as basis weight, tensile strength, bending stiffness, liquid permeability, breathability and/or hydrophilicity.

The basis weight of the first layer and the basis weight of the second layer may be at least 1 g/m², or at least 2 g/m², or at least 3 g/m², or at least 5 g/m²; and the basis weight may further be not more than 1000 g/m², or not more than 500 g/m², or not more than 200 g/m², or not more than 100 g/m², or not more than 50 g/m², or not more than 30 g/m².

The tensile strength of the first and second layers may be at least 3 N/cm, or at least 4 N/cm, or at least 5 N/cm. The tensile strength may be less than 100 N/cm, or less than 80 N/cm, or less than 50 N/cm, or less than 30 N/cm, or less than 20 N/cm.

The bending stiffness of the first layer and the bending stiffness of the second layer may be at least 0.1 mNm, or at least 0.2 mNm, or at least 0.3 mNm. The bending stiffness may be less than 200 mNm, or less than 150 mNm, or less than 100 mNm, or less than 50 mNm, or less than 10 mNm, or less than 5 mNm.

Generally, the higher the tensile strength and the bending stiffness of the first and second layer, the more rigid, but also the more stable the overall structure will become. Hence, the choice of tensile strength and bending stiffness for the first and second layer depends on the application of the structure, balancing overall softness, drape and conformability requirements with overall stability and robustness.

The first and second layers (110, 120) are connected to each other via ligaments (130).

In the structure (100), each of the first and second layers (110, 120) has an inner (111, 121) and an outer surface (112, 111) wherein the inner surfaces (111, 121) face towards the ligaments (130). Each of the first and second layer (110, 120) in the structure (100) further has a longitudinal dimension which is parallel to the longitudinal dimension of the structure (100) and which is confined by two spaced apart lateral edges (114, 124). Each of the first and second layers (110, 120) also has a lateral dimension parallel with the lateral dimension of the structure and confined by two spaced apart longitudinal edges. The first and second layer (110, 120) in the structure (100) overlap at least in the area where the ligaments are positioned between the first and second layer. The first and second layer (110, 120) may also overlap—at least partly—in the areas extending outboard of the area where the ligaments (130) are positioned.

The ligaments (130) are positioned in between the first and second layer (110, 120). Each ligament (130) has a longitudinal dimension confined by two spaced apart lateral edges (134) and a lateral dimension confined by two spaced apart longitudinal edges.

In FIG. 1A, a coordination system is shown with X-, Y- and Z-directions. The longitudinal dimension of the overall structure (100) and of the first and second layer (110, 120) extends along the longitudinal direction X of the coordination system. The longitudinal dimension of the ligaments (130) in the structure's initial flat configuration may substantially extend along the longitudinal direction X of the illustrated coordination system.

Likewise, the lateral dimension of the overall structure (100) and of the first and second layer (110, 120) extends along the lateral direction Y of the coordination system. The lateral dimension of the ligaments (130) in the structure's initial flat configuration may substantially extend along the lateral direction Y of the illustrated coordination system.

The caliper of the structure (100) extends along the Z direction of the coordination system.

Each ligament (130) is attached to the inner surface (111) of the first layer (110) at or adjacent to one of the lateral edges (134) of the respective ligament. Each ligament (130) is also attached to the inner surface (121) of the second layer (120) at or adjacent to the other lateral edge (134) of the respective ligament. Due to this attachment, the ligaments (130) will generally adopt a C-like, Z-like, T-like, or Double-T-like shape when the structure is in its erected configuration.

When the ligament adopts a Z-like shape when the structure is in its erected configuration, the ligament (130) is attached to the inner surface (111) of the first layer (110) with a portion of the ligament's first surface (131) at or adjacent to one of its lateral ligament edges (134) in the first ligament attachment region (135) and is further attached to the inner surface (121) of the second layer (120) with a portion of the ligament's second surface (132) at or adjacent to its other lateral ligament edge (134) in the second ligament attachment region (136).

Moreover, for Z-like shapes, the ligament's (130) first surface (131) may not be facing towards the inner surface (121) of the second layer (110) when the structure (100) is in its initial flat configuration, and the ligament's second surface (132) may not be facing towards the inner surface (111) of the first layer (120) when the structure (100) is in its initial flat configuration. With such embodiments, it is possible to obtain structures with no folds and hinges (or very few folds and hinges, e.g. when the ligaments have different longitudinal dimension in their free intermediate portion (137), see below). Hence, these structures will generally exhibit a lower tendency to partly erect in the absence of an applied force along the longitudinal dimension and will consequently remain in their initial flat configuration. Also, such structures will generally exhibit a greater tendency to return to their initial flat configuration when the applied force is released.

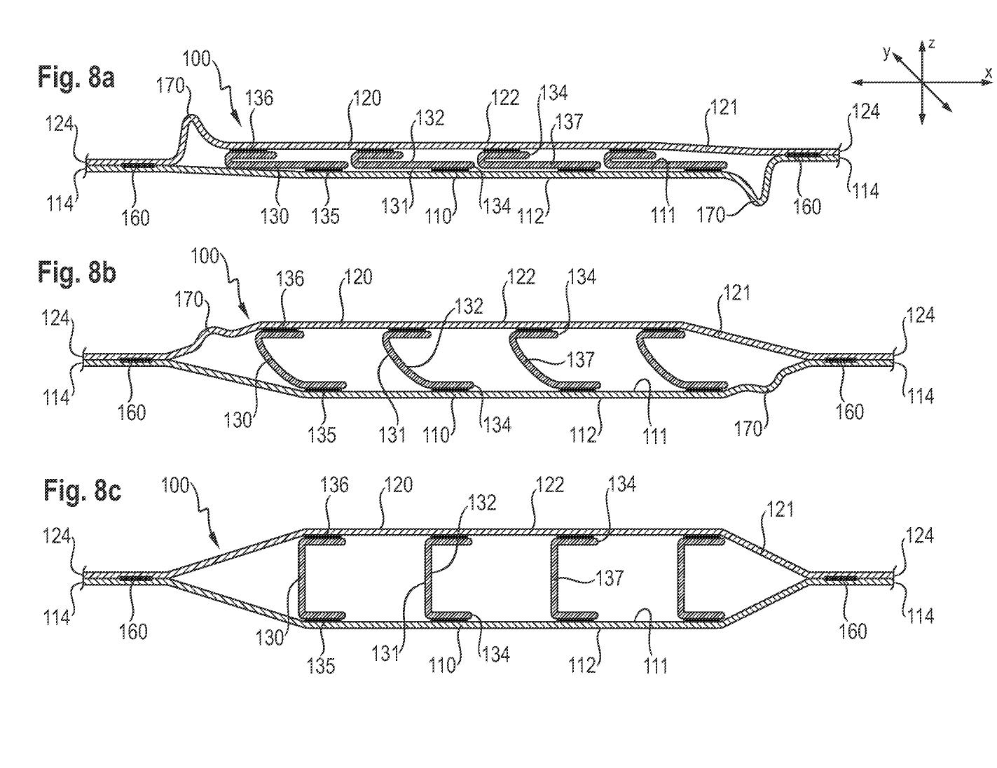

Alternatively, when the ligament adopts a C-like shape when the structure is in its erected configuration (as exemplary shown in FIG. 8A through 8C), the ligament (130) is attached to the inner surface (111) of the first layer (110) with a portion of the ligament's first surface (131) at or adjacent to one of its lateral ligament edges (134) in the first ligament attachment region (135) and is further attached to the inner surface (121) of the second layer (120) with a portion of the ligament's first surface (132) at or adjacent to its other lateral ligament edge (134) in the second ligament attachment region (136).

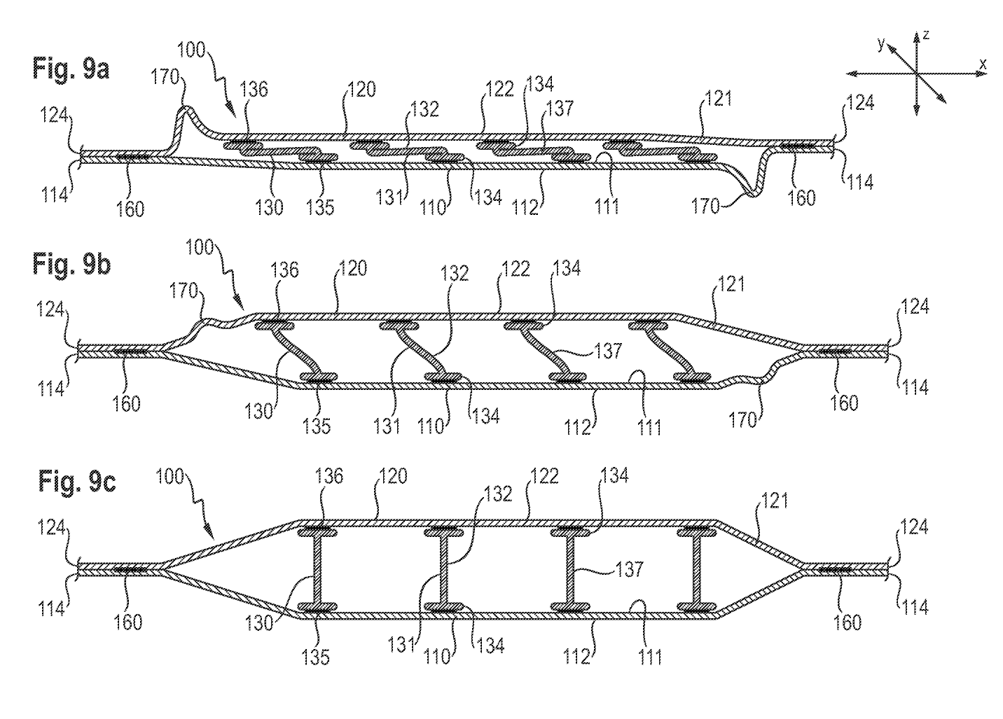

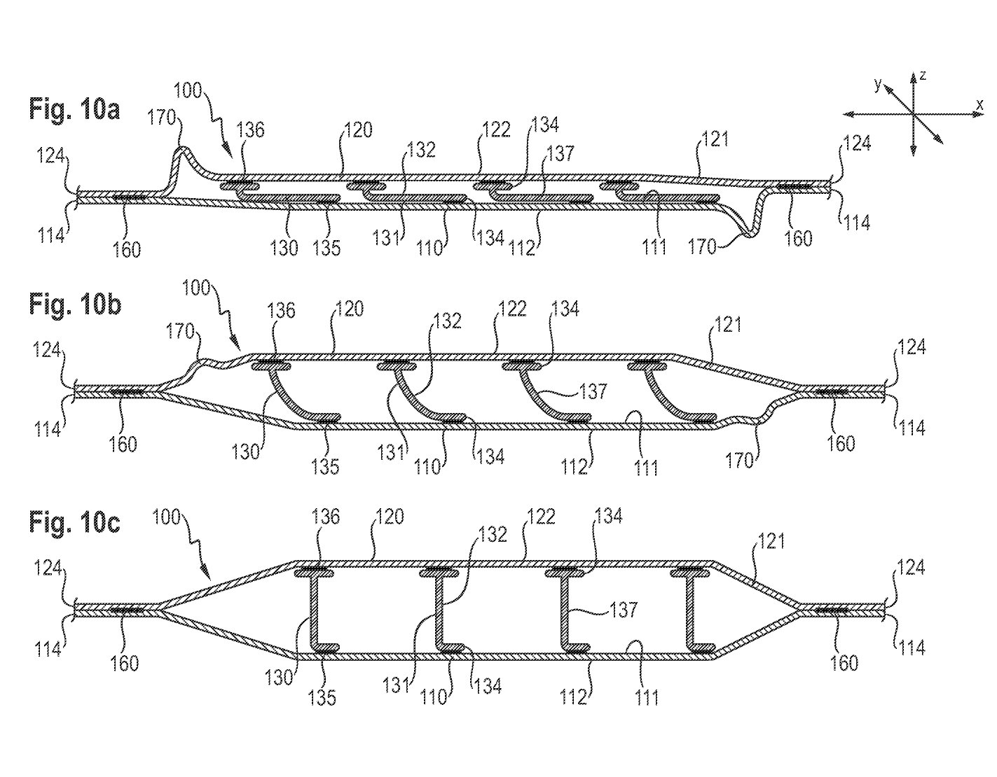

Still alternatively, one portion of the ligament at or adjacent to one of its lateral ligament edges may be attached to the first layer such as to form a T-like (as exemplary shown in FIG. 10A through 10C), or Double-T-like shape (as exemplary shown in FIG. 9A through 9C). To facilitate such attachment, the portion of the ligament adjacent to a first lateral ligament edge has to comprise at least two ligament layers and the two ligament layers are not attached to each other in the portion of the ligament which is attached to the first layer. Thereby, the ligament can be split up and unfolded such that both ligament layers can be respectively attached to the first layer in the first ligament attachment region (135) to form a T-like shape when the structure is in its erected configuration.

For embodiments where a ligament adopts a T-like shape, the portion of the ligament at or adjacent to the second lateral ligament edge is attached to the second layer with either its first or second surface to form the second ligament attachment region (136). If the ligament is also made of a laminate in the second ligament attachment region (136), the ligament layers are not split up in this area (i.e. only the portion adjacent to the first lateral ligament edge forms a T-like shape).

When the ligament adopts a Double-T-like shape when the structure is in its erected configuration, the portion of the ligament at or adjacent to the second lateral ligament edge is attached to the second layer similar to the manner in which the portion at or adjacent to the first lateral ligament edge is attached to the first layer, i.e. the ligament laminate is split up and unfolded such that two ligament layers can be respectively attached to the second layer to form the second ligament attachment region (136).

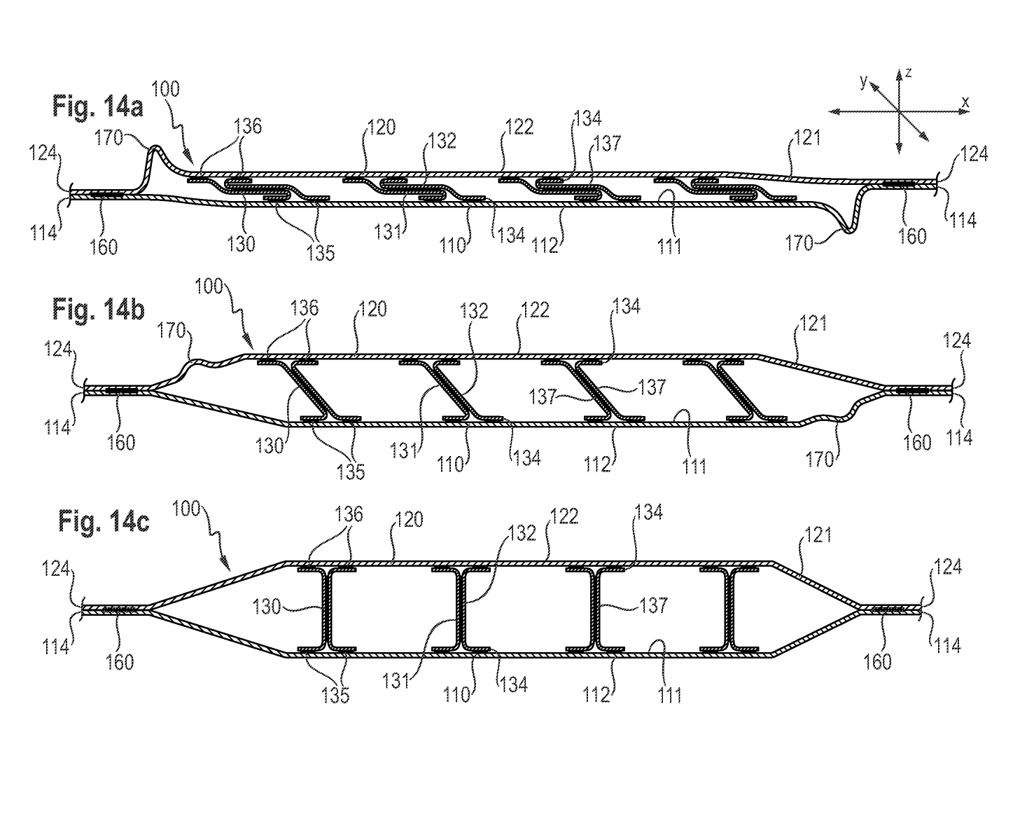

Splitting up and unfolding the ligament layers to form the respective first and second ligament attachment regions (135, 136) is exemplified in FIGS. 14A through 14C (showing ligaments with Double-T-like shape)

In a given structure, all ligaments may adopt a Z-like shape when the structure is in its erected configuration. Alternatively, all ligaments may adopt a C-like shape, all ligaments may adopt a T-like shape or all ligaments may adopt a Double-T-like shape when the structure is in its erected configuration. In a further alternative, a given structure may have a combination of ligaments selected from the group consisting of: ligaments adopting a Z-like shape, C-like shape, T-like shape and Double-T-like shapes.

In a given structure, ligaments adopting a Z-like shape when the structure is in its erected configuration may all adopt the same orientation. That means, in such embodiments none of the ligaments adopting a Z-like shape has an inverse configuration of another ligament adopting a Z-like shape.

Likewise, in a given structure, ligaments adopting a C-like shape when the structure is in its erected configuration may all adopt the same orientation. That means, in such embodiments none of the ligaments adopting a C-like shape has an inverse configuration of another ligament adopting a C-like shape.

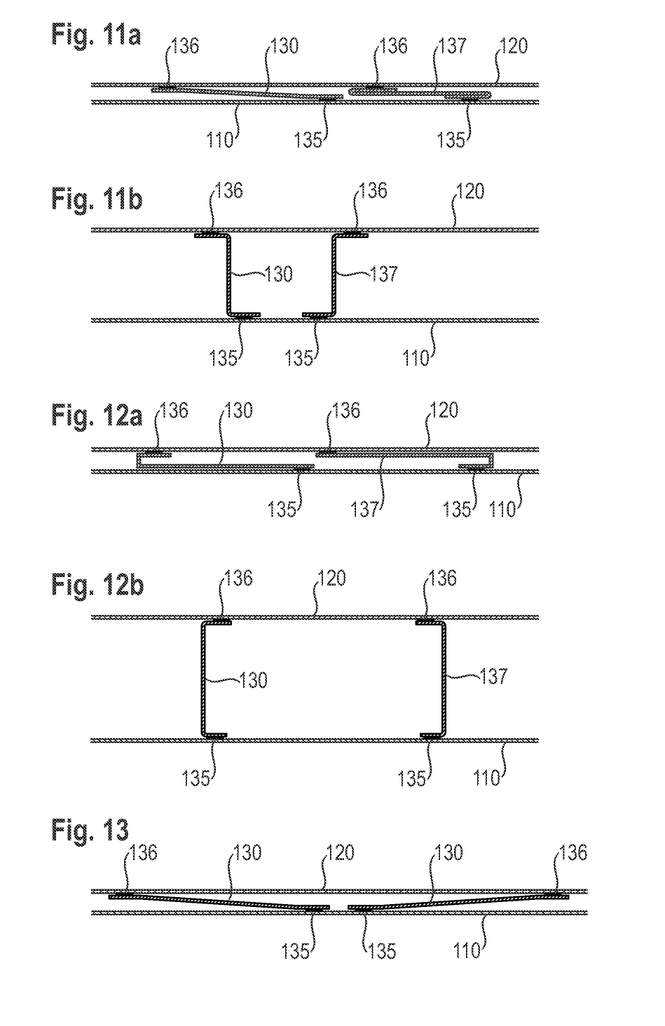

However, it is also possible to facilitate the structure such that that one or more ligaments adopt a Z-like shape which has an inverse configuration of one or more other ligaments with Z-like shape. An example of such structure is illustrated in FIG. 11A (flat configuration) and FIG. 11B (erected configuration).

Similarly, a structure with one or more ligaments adopting a C-like shape which has an inverse configuration of one or more other ligaments with C-like shape is exemplified in FIG. 12A (flat configuration) and FIG. 12B (erected configuration).

Such inverse configurations are feasible as long as the structure can be readily converted from its flat configuration to its erected configuration. An example of a structure having Z-like ligaments with inverse configuration which cannot be converted from its flat to erected configuration is shown in FIG. 13. In this structure, the ligament on the right side of the Figure “blocks” the ligament on the left side of the Figure from being erected upon application of a force along the longitudinal direction (and vice versa, the ligament on the left side of the Figure “blocks” the ligament on the left side of the Figure from being erected). The ligament shown on the right side (or left side) of the Figure also cannot serve as a stop aid (explained below in detail) as no leeway is provided.

Generally, ligaments in a given structure have to be configured and attached accordingly such that the structure is able to be converted from an initial flat configuration into an erected configuration whereby the ligaments convert from an initial flat configuration into an erected configuration. Moreover, the ligaments in a given structure have to be configured and attached accordingly such that, upon further application of a force along the longitudinal dimension, it would be possible—in the absence of a means that maintains the structure in its erected configuration, such as a stop aid, which is described below—to convert, the erected structure into a turned-over flat structure, wherein the ligaments would have been turned over by 180° based on the ligament's position in the initial flat structure configuration.

The longitudinal dimension of each ligament (130) between the first and second ligament attachment regions (135, 136) remains unattached to the first and second layer (110, 120) or is releasable attached to the first and/or second layers (110, 120) and/or to their neighboring ligament(s). This unattached or releasable attached portion is referred to as the “free intermediate portion” (137) of the ligament (130). “Releasable attached” means a temporary attachment to the first and/or second layer (110, 120) and/or the neighboring ligament(s) in a way, that the bond strength is sufficiently weak to allow easy detachment from the first and/or second layer and/or the neighboring ligament(s) upon initial elongation of the structure (100) along the longitudinal dimension without rupturing or otherwise substantially damaging the ligaments (130) and/or the first and/or second layer (110, 120) and without substantially hindering the conversion of the structure from its initial flat configuration into its erected configuration. Such releasable attachments may help to maintain the structure in its initial flat configuration, e.g. during manufacturing processes when the structure is incorporated into an article.

When the structure is in its initial flat configuration, the first surface (131) of a ligament's free intermediate portion (137) faces towards the first layer (110) and the second surface (132) of a ligament's free intermediate portion (137) faces towards the second layer (120). When the structure (100) is converted into its erected configuration, the first surface (131) of a ligament's (130) free intermediate portion (137) faces towards the second surface (132) of its neighboring ligament (130). This is irrespective as to whether the ligament has been attached to adopt a Z-like, C-like, T-like or Double-T-like shape. Unless expressly mentioned herein, neighboring ligaments refers to ligaments which are neighboring along the longitudinal dimension.

The ligaments (130) may be attached to the first and second layer (110, 120) by any means known in the art, such as by use of adhesive, by thermal bonding, by mechanical bonding (such as pressure bonding), by ultrasonic bonding, or by combinations thereof. The attachment of the ligaments to the first and second layer is permanent, i.e. the attachment should not be releasable by forces which can typically be expected during use of the structure.

Structures (100) as described supra are able to adopt an initial flat configuration when no external forces are applied. Upon application of a force along the longitudinal dimension, the structure will not only increase its longitudinal dimension, i.e. get longer, but simultaneously, the structure will also increase in caliper, i.e. in the direction perpendicular to the longitudinal and lateral dimension. Moreover, such structures typically do not exhibit necking upon elongation, i.e. the lateral dimension does not decrease.

Such structures may also return to essentially their initial longitudinal dimension and (flat) caliper upon release of the external force applied along the longitudinal dimension.

The force along the longitudinal dimension may be applied e.g. by grabbing the structure adjacent to the lateral edges (114, 124) of the first and second layer (110, 120) (outside the area, where the ligaments (130) are positioned). The force may also be applied indirectly, i.e. without grabbing the structure, when the structure is built into an absorbent article, such as a disposable diaper or pant.

The structure can be facilitated such that, in its initial flat configuration, no hinges and folds may be created in the structure (100) and the first and second layers (110, 120) as well as the ligaments (130) lie flat and outstretched (however, not extended beyond their dimension in the relaxed state). Such structures allow having a very thin caliper in the flat configuration. In these kinds of structures, all ligaments will adopt a Z-like shape when the structure is in its erected configuration. Moreover, due to the absence of any folds and hinges in the initial flat configuration such structures will not exhibit any tendency to turn into a (partly) erected configuration when they are in the initial flat configuration without the application of an external force. Thus, they will more readily remain in a complete initial flat configuration with no external forces applied compared to structures with folds and hinges, which may exhibit some tendency to partly erect on their own motion depending on the properties of the materials selected for the first and second layer and especially depending on the properties of the materials selected for the ligaments (such as bending stiffness).

In structures where no hinges may be created, the complete first surface (131) of each ligament (130) (i.e. not only the free intermediate portion) does not face towards the inner surface (121) of the second layer (110) when the structure (100) is in its initial flat configuration, and the complete second surface (132) of each ligament (130) (i.e. not only the free intermediate portion) does not face towards the inner surface (111) of the first layer (120) when the structure is in its initial flat configuration. Such a structure is for example illustrated in FIG. 1A/1B.

FIGS. 7 and 8 show structures where the ligaments are folded when the structure is in its initial flat configuration.

Also, for certain other executions, e.g. those where the longitudinal dimension of the free intermediate portion (137) of the ligaments (130) differs from each other, the initial flat configuration may have some folds and hinges.

Upon application of a force along the longitudinal dimension of the structure (100), the first and second layer (110, 120) shift relative to each other in opposite longitudinal directions such that the structure length extends. At the same time, the structure (100) erects due to the erection of the ligaments (130). Between neighboring ligaments, a space, a so-called “cell” (140) is formed, which is confined by the first and second layer and the respective neighboring ligaments. When viewed from the side, along the lateral direction, the cells may take for example a rectangular shape, a trapezoid shape, a rhomboid shape, or the like.

For structures wherein the longitudinal dimension of the free intermediate portion (137) is the same for all ligaments, the structure (100) will adopt its highest possible caliper when the ligaments (130) are in an upright position, i.e. when the free intermediate portion (137) of the ligaments (130) is perpendicular to the first and second layers (110, 120) between neighboring ligaments. However, the formation of this upright position may possibly be hindered, at least in some areas, when a force towards the caliper of the structure is applied at the same time, if this force is sufficiently high to deform the structure in the caliper dimension.

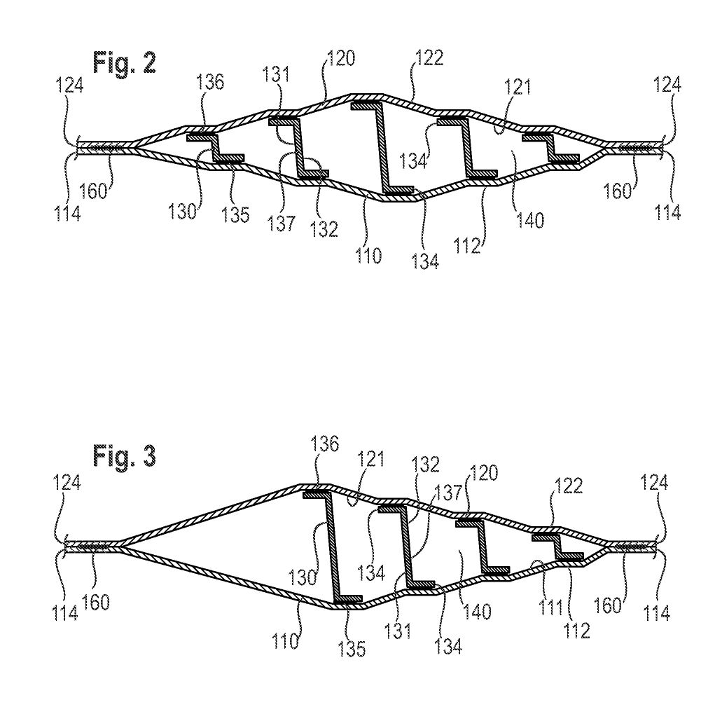

In structures, where the longitudinal dimension of the free intermediate portion (137) differs between different ligaments (130), the ligaments (130) may not be perpendicular to the first and second layer (110, 120) in the erected configuration, see e.g. FIGS. 2 and 3). In such embodiments, the first and second layer (110, 120) are not parallel to each other when the structure is in its erected configuration but instead, the first and/or second layer (110, 120) take(s) an inclined shape.

Tensile strength, and especially bending stiffness, impacts the resistance of the structure (especially of the structure in its erected configuration) against compression forces. Thus, when the ligaments have a relatively high tensile strength and bending stiffness, the structure is more resistant to forces applied in the Z-direction (i.e. towards the caliper of the structure). Also, if the first and/or second layers have relatively high tensile strength and relatively high bending stiffness, resistance of the structure against forces applied in the Z-direction (i.e. towards the caliper of the structure) is increased. For the present invention, the compression resistance of the erected structure is measured in terms of the structure's modulus according to the test method set out below.

As a difference in bending stiffness results in a difference in the resistance against compression forces (and hence, the modulus) applied in the Z-direction (i.e. towards the caliper of the structure), using ligaments with different bending stiffness enables structures which have improved resistance to compression (higher modulus) in the Z-direction in areas where the ligaments have higher bending stiffness, whereas the structure adjusts more readily e.g. to curved surfaces in the areas where ligaments with lower bending stiffness are applied (lower structure modulus). For example, the bending stiffness of the ligaments which are arranged in the center of the structure along the longitudinal dimension may be higher compared to the ligaments arranged towards the lateral edges of the structure.

In addition to the tensile strength and the bending stiffness of the materials used for the structure, the resistance of the (erected) structure against compression forces is also impacted by the number of ligaments which are provided, and the distance between neighboring ligaments. Neighboring ligaments which have a relatively small gap between them along the longitudinal dimension of the structure will provide for higher resistance of the erected structure against compression forces (higher modulus) compared to a structure wherein neighboring ligaments are more widely spaced apart along the longitudinal dimension of the structure (lower modulus) as long as the material used for the different ligaments and their size does not differ from each other.

Moreover, if the modulus is measured between neighboring ligaments, the modulus will typically be lower than the modulus measured in the location where a ligament is positioned.

Modulus is measured following the test method set out below and is measured in the Z-direction of the structure.

The structure in its erected configuration may have a modulus of at least 0.004 N/mm², or at least 0.01 N/mm², or at least 0.02 N/mm², or at least 0.03 N/mm² in those areas where a ligament is posited as well as in the areas between neighboring ligaments.

Moreover, for certain applications, it may also be desirable to avoid excessively high compression resistance (i.e. too high modulus), e.g. to avoid that the erected structure is too stiff. This may be preferred when certain conformity of the structure to a surface (such as skin) is desirable. For such structures, the structure in its erected configuration may have a modulus of not more than 1.0 N/mm², or not more than 0.5 N/mm², or not more than 0.2 N/mm², but at least 0.1 N, or at least 0.5 N, or at least 1.0 N in those areas where a ligament is posited as well as in the areas between neighboring ligaments.

Alternatively, the structure in its erected configuration may have a modulus of at least 0.05 N/mm², or at least 0.08 N/mm², or at least 0.1 N/mm², or at least 0.13 N/mm², but not more than 2.0 N/mm², or not more than 1.0 N/mm², or not more than 0.5 N/mm², or not more than 0.3 N/mm² in the locations where the ligaments are positioned, while the structure in its erected configuration may have a modulus of at least 0.004 N/mm², or at least 0.01 N/mm², or at least 0.02 N/mm², or at least 0.03 N/mm² between neighboring ligaments.

Generally, the ligaments (130) may be spaced apart from each other along the longitudinal dimension at equal distances or, alternatively, at varying distances. Also, the ligaments (130) may be spaced apart from each other such, that the ligaments (130) do not overlap with each other when the structure (100) is in its initial flat configuration. Thereby, it is possible to provide structures (100) with very small caliper when the structure (100) is in its initial flat configuration, as the ligaments (130) do not “pile up” one on top of each other when the structure is in its initial flat configuration.

The free intermediate portion (137) of the ligaments (130) may all have the same longitudinal dimension. Thereby, the structure will have a constant caliper across its longitudinal (and lateral) dimension when the structure (100) is in its erected configuration (except for the areas longitudinally outward of the regions where the ligaments are placed, towards the lateral edges of the structure). An example of such an embodiment is shown in FIG. 1B. Alternatively, the longitudinal dimension of the free intermediate portion (137) may vary for different ligaments (130) in a structure (100). Thereby, the caliper of the structure will vary across the longitudinal dimension. Examples of such embodiments are shown in FIGS. 2 and 3. The free intermediate portion (137) of neighboring ligaments (130) may increase or decrease along the longitudinal dimension of the structure, or the free intermediate portion (137) may vary randomly along the longitudinal dimension, depending on the desired shape of the structure in its erected configuration and on the intended use of the structure.

As exemplified in FIG. 2, the one or more ligaments (130) in the center of the structure (as seen along the longitudinal dimension) may have a longer free intermediate portion (137) than the ligaments towards the lateral edges of the structure, resulting in a structure with a higher caliper in the center than towards the edges when the structure is in its erected configuration. Thereby, the resulting erected structure may, for example, adopt a rhomboid or trapeze shape (when viewed from the side) Also, one or more ligaments (130) towards one of the lateral edges may have a longer free intermediate portion (137) than one or more ligaments towards the other lateral edge, resulting in a structure with a wedge-like shape (when viewed from the side) when the structure is in its erected configuration. An example of such an embodiment is illustrated in FIG. 3. Generally, the caliper of the erected structure depends on the length of the free intermediate portion (137) of the ligaments (130).

Generally, the maximum increase in caliper of the structure in its erected configuration (versus the caliper of the structure in its initial flat configuration) depends to the longitudinal dimension of the free intermediate portion (137) of the ligaments (130)—less the caliper of the ligament. If the ligaments (130) differ from each other in the longitudinal dimension of their free intermediate portions (137), the maximum increase in caliper of the structure in its erected configuration, as used herein, is based on the longitudinal dimension of the ligament with the largest longitudinal dimension of free intermediate portion. If a stop aid (160, 180, 190) is used (as described below), the structure may not be able to adopt its maximum increase in caliper in its erected configuration as the erection is stopped by the stop aid before the maximum erection, which would have been possible in the absence of a stop aid, is reached.

The maximum increase in caliper of the erected structure versus the structure in its initial flat configuration may be at least 2 mm, or at least 3 mm, or at least 4 mm, or at least 5 mm, or at least 7 mm, or at least 10 mm, and may be less than 100 mm, or less than 70 mm, or less than 50 mm, or less than 40 mm, or less than 30 mm, or less than 25 mm, or less than 20 mm, or less than 15 mm, or less than 10 mm, or less than 5 mm, wherein the maximum increase in caliper is solely defined by the longitudinal dimension of the free intermediate portion. As used herein, the maximum increase in caliper is equal to the longitudinal dimension of the free intermediate portion.

As the caliper of the structure in its flat configuration inter alia depends on the caliper of the ligaments, (though the caliper of the ligaments will be significantly smaller that the longitudinal dimension of the free intermediate portion), for the present invention, the caliper of the ligament (with the longest longitudinal dimension of the free intermediate portion) is subtracted from the longitudinal dimension of the free intermediate portion when defining the maximum increase in caliper. The caliper of the ligament is measured according to the test method set out below.

If formation of wrinkles in the first and second layer (110, 120) and/or in the ligament (130) shall be avoided when the structure is in its erected configuration, it is desirable, that the ligaments (130) are arranged such that, when the structure is in its initial flat configuration, the longitudinal dimension of the ligaments is substantially parallel with the longitudinal dimension of the first and second layer. “Substantially parallel” means that the orientation of the longitudinal dimension of the ligaments does not deviate by more than 20°, or not more than 10°, or not more than 5°, or not more than 2° from the longitudinal dimensions of the first and second layer. The orientation of the longitudinal dimension of the ligaments may also not deviate at all from the longitudinal dimension of the first and second layer.

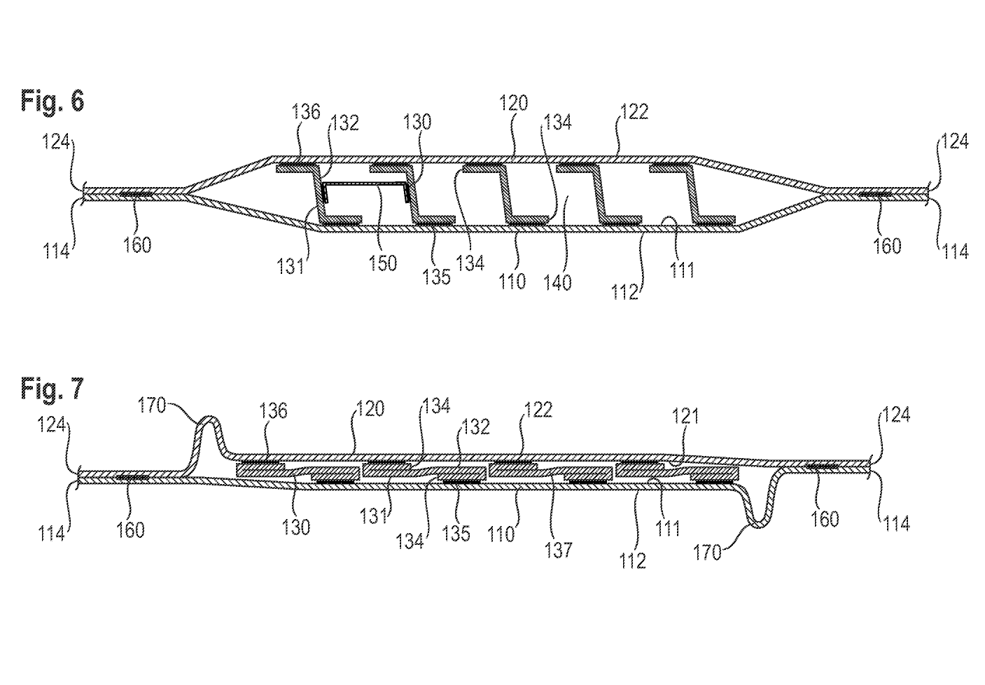

Typically, the free intermediate portions (137) are not attached to each other. However, for certain applications it may be desirable that the free intermediate portion (137) of neighboring ligaments (130) are attached to each other directly, which results in some buckling of the ligaments attached to each other when the structure is in its erected configuration. Alternatively, the free intermediate portion (137) of neighboring ligaments (130) may be attached to each other indirectly via a separate ligament-to-ligament material (150), such as a piece of nonwoven, film, paper, or the like (shown in FIG. 6). If neighboring ligaments are attached to each other, especially via a separate piece of material, the overall stability and stiffness of the structure may be improved. Also, in such embodiments, the cells formed between neighboring ligaments when the structure is in its erected configuration, are separated into sub-cells.

Depending on the materials used for the structure, especially depending on the material used for the ligaments (130) and the manner in which the ligaments are attached to the first and second layer (110, 120), the erected structure may or may not return substantially completely to its initial flat configuration upon release of the force applied along the longitudinal dimension, as can be determined when a structure has been erected to adopt the maximum possible caliper and has been held in this position for 5 minutes and immediately after it is allowed to relax for 1 minute upon release of the force applied along the longitudinal direction.

Generally, the first and second layer (110, 120) may have the same lateral dimension and the longitudinal edges of the first and second layer may be congruent with each other. Also, the lateral dimension of all ligaments (130) may be the same, and the lateral dimension of all ligaments (130) may also be the same as the lateral dimension of the first and second layer (110, 120). The longitudinal edge of the first layer (110) may coincide with the longitudinal edge of the second layer (120) and/or with the longitudinal edges of the ligaments (130),

Also, one or more of the ligaments (130) may have the same lateral dimension as the first and second layer (110, 120) and one or more of the ligaments, which do not have the same lateral dimension as the first and second layer may be flanked on one or both longitudinal edges by other ligaments, such that the structure has laterally neighboring ligaments.

Generally, the structure may have a longitudinal and/or lateral dimension of at least 4 cm, or at least 5 cm, or at least 6 cm, or at least 7 cm and may have a longitudinal and/or lateral dimension of not more than 100 cm, or not more than 50 cm, or not more than 30 cm, or not more than 20 cm. If the longitudinal dimension is not the same along the lateral direction, the minimum longitudinal dimension is determined at the location where the longitudinal dimension has its minimum and the maximum longitudinal dimension is determined at the location where the longitudinal dimension has its maximum. Similarly, if the lateral dimension is not the same along the longitudinal direction, the maximum lateral dimension is determined at the location where the lateral dimension has its maximum and the minimum lateral dimension is determined at the location where the lateral dimension has its minimum. The structure may have an overall rectangular shape.

The longitudinal and lateral dimensions are determined when the structure is in its initial flat configuration.

The free intermediate portion (137) of the ligaments (130) may have a longitudinal dimension of at least 2 mm, or at least 3 mm, or at least 4 mm, or at least 5 mm, or at least 7 mm, or at least 10 mm, and may have a longitudinal dimension of less than 100 mm, or less than 70 mm, or less than 50 mm, or less than 40 mm, or less than 30 mm, or less than 25 mm, or less than 20 mm, or less than 15 mm, or less than 10 mm, or less than 5 mm.

### Ligaments

The ligaments may be elastic, non-elastic or highly non-elastic. Also, the ligaments may be extensible, non-extensible or highly non-extensible. The ligaments are made of nonwovens, film, paper, sheet-like foam, woven fabric, knitted fabric, or combinations of these materials. Combinations of these materials may be laminates, e.g. a laminate of a film and a nonwoven.

The ligaments within a structure may all be made of the same material or, alternatively, the different ligaments within a structure may be made of different materials.

The material may be the same throughout each ligament, i.e. the ligament may be made of a single piece of material or of a laminate wherein each laminate layer extends over the complete ligament.

The ligaments may have the same properties throughout the ligament, especially with regard to bending stiffness and tensile strength.

Alternatively, the ligaments may have areas with properties (such as bending stiffness and/or tensile strength) which differ from the properties in one or more other areas of a given ligament.

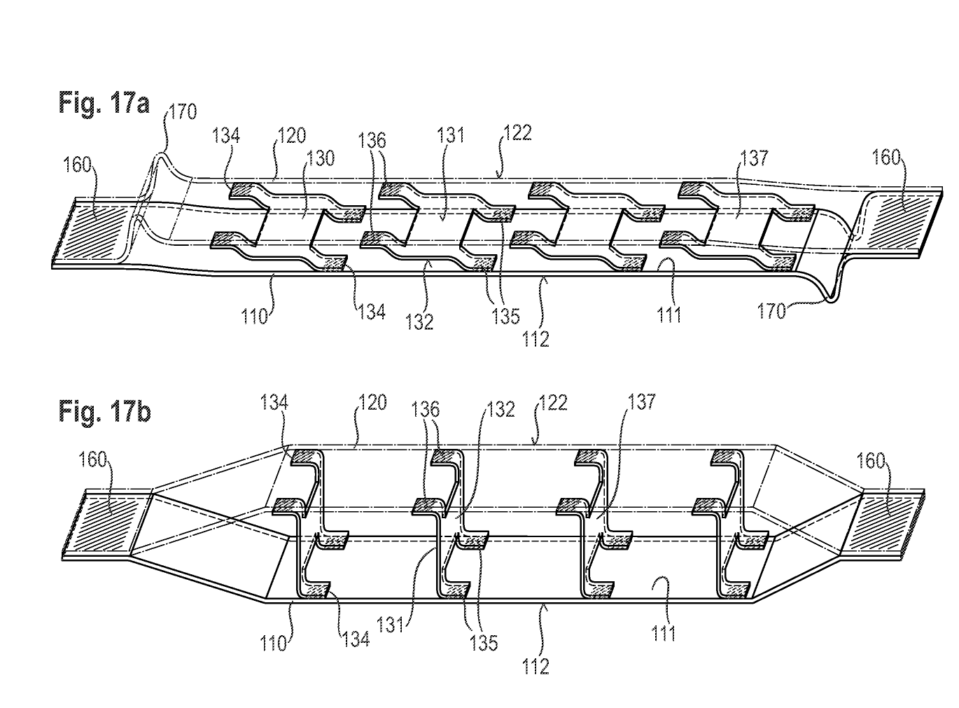

Such areas with differing properties can be facilitated by modifying the material in one or more ligament areas, e.g. by mechanical modification. Non-limiting examples of mechanical modifications are the provision of cut outs in one or more areas of the ligament to reduce tensile strength and bending stiffness in those areas; incremental stretching (so-called “ring-rolling”) one or more ligament areas to reduce tensile strength and bending stiffness; slitting one or more ligament areas to reduce tensile strength and bending stiffness; applying pressure and/or heat to one or more areas of ligaments; or combinations of such mechanical modifications. Application of heat and/or pressure may either increase or reduce tensile strength and bending stiffness. For example, if heat and/or pressure are applied on a ligament made of nonwoven with fibers made of thermoplastic material, the fibers may be molten together and bending stiffness and tensile strength can be increased. However, if an excessive amount of heat and/or pressure is used, the material of the ligaments may be damaged (such as fiber breakage in a nonwoven web) and weakened areas are formed, thus reducing bending stiffness and tensile strength. Cutting out areas of the ligaments may either result in the formation of apertures within the ligament or the cut out may not be fully surrounded by uncut areas as is illustrated in FIGS. 17A (essentially flat configuration) and 17B (erected configuration).

Alternatively, or in addition to the above, areas with different properties can also be obtained by chemically modifying one or more areas of the ligament, e.g. by adding chemical compounds, such as binders or thermoplastic compositions to increase bending stiffness and tensile strength, winch may be followed by curing. One or more areas with different properties can also be obtained by providing different materials in different areas or by adding additional pieces of materials only in certain areas (thus, e.g. forming a laminate in these areas), while having another material (which may be the same or different from the piece of material added in certain areas) which is coextensive with the ligament and used throughout the ligament.

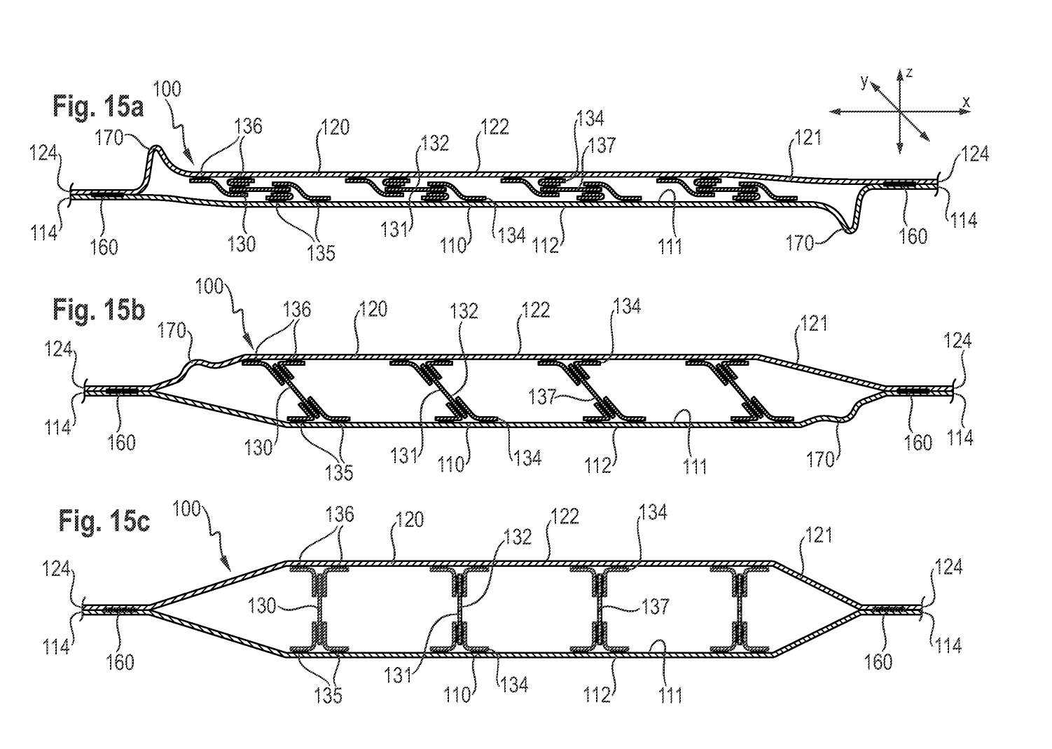

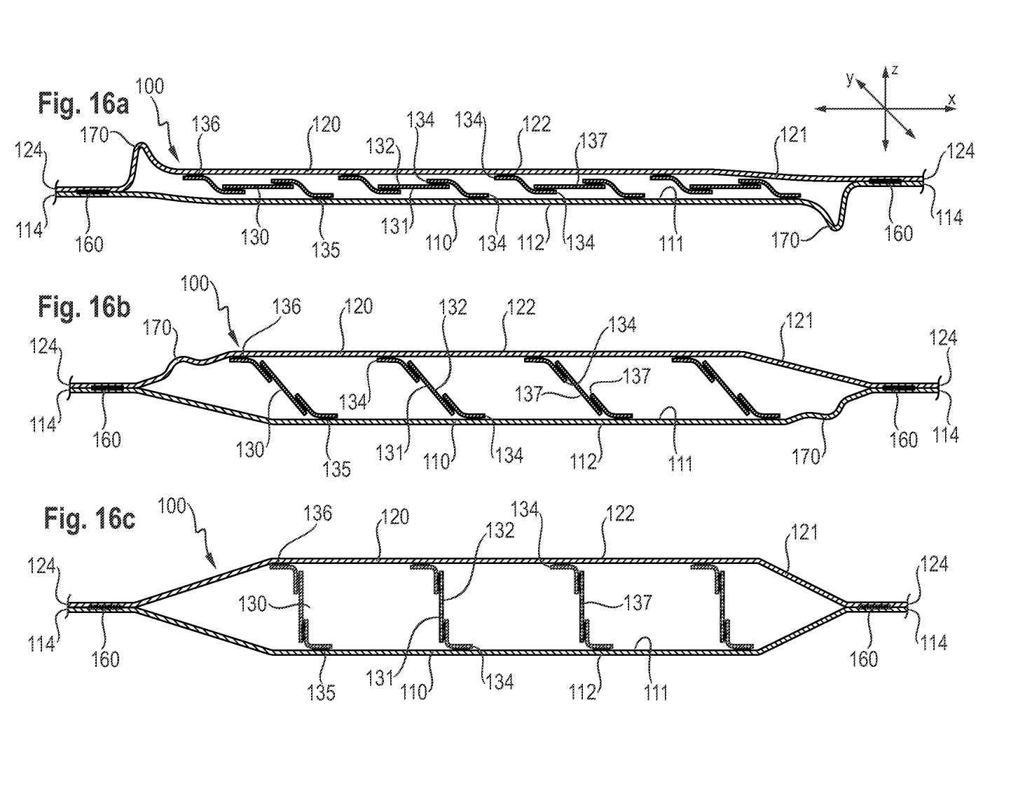

Still further, one or more areas with different properties may be achieved by providing ligaments which are assembled by attaching different pieces of material to each other so they overlap partly and partly do not overlap, such that together they form the overall ligament (instead of having one continuous material coextensive with the ligament to which additional pieces of materials) are added only in certain areas). Examples of structures with ligaments being assembled of pieces of (different) materials are illustrated in FIGS. 15A through 15C and 16A through 16C.

By having ligaments with one or more areas having properties different from the remaining ligament, the behavior of the structure with respect to e.g. bending stiffness and tensile strength can be fine tuned to meet certain needs in different areas of the structure (e.g. the ability to accommodate readily and softly to a surface such as the skin of a wearer in some areas and to be stiffer and more resistant to compression in other areas to close gaps).

If providing such areas of different properties by any of the above means, it may be especially desirable to provide them in the areas of the free intermediate portion of a ligament which are directly adjacent to the first and second ligament attachment region. The areas of the free intermediate portion which are directly adjacent to the first and second ligament attachment regions are those areas which bend upon application of a force along the longitudinal direction of the structure, thus erecting the free intermediate portion.

For example, by having higher bending stiffness and/or tensile strength in the areas of the tree intermediate portion directly adjacent to the first and second ligament attachment regions, results in ligaments which have a higher tendency to convert back from the erected configuration to the initial flat configuration upon relaxation of the force applied along the longitudinal dimension.

Alternatively, having lower bending stiffness and/or tensile strength in the areas of the free intermediate portion directly adjacent to the first and second ligament attachment regions, results in ligaments which have a lower tendency to convert back from the erected configuration to the initial flat configuration upon relaxation of the force applied along the longitudinal dimension (i.e. have a higher tendency to remain erected or at least partly erected). Such structures would also require less force to be converted into their erected configuration, as the ligament's free intermediate portion would bend more readily in the areas directly adjacent to the first and second ligament attachment regions.

The basis weight of each of the ligaments may be at least 1 g/m², or at least 2 g/m², or at least 3 g/m², or at least 5 g/m²; and the basis weight may further be not more than 1000 g/m², or not more than 500 g/m², or not more than 200 g/m², or not more than 100 g/m², or not more than 50 g/m², or not more than 30 g/m².

If the tensile strength is the same throughout the ligament, the tensile strength of the ligaments may be at least 3 N/cm, or at least 5 N/cm or at least 10 N/cm. The tensile strength may be less than 100 N/cm, or less than 80 N/cm, or less than 70 N/cm, or less than 50 N/cm, or less than 40 N/cm.

If the tensile strength is the same throughout the ligament, the bending stiffness of the ligaments may be at least 0.1 mNm, or at least 0.2 mNm, or at least 0.3 mNm. The bending stiffness may be less than 500 mNm, or less than 300 mNm, or less than 200 mNm, or less than 150 mNm. Principally, for the ligaments the same considerations regarding overall softness, drape and conformability versus overall stability and robustness apply as set out above for the first and second layer. However, the bending stiffness and tensile strength of the ligaments typically has a higher impact on the overall bending resistance of the erected structure (when a force is applied along the caliper of the structure, i.e. perpendicular to the lateral and longitudinal dimension of the structure) vs. the impact of the bending stiffness and tensile strength of the first and second layer. Thus, it may be desirable that the ligaments have a higher bending stiffness and a higher tensile strength than the first and second layer.

The different ligaments in a structure may vary from each other in basis weigh and/or tensile strength, bending stiffness and the like.

### Stop Aid

It may be desirable to define a maximum shifting of the first and second layers (110, 120) relative to each other in opposite longitudinal directions upon application of the force along the longitudinal dimension of the structure (100). This can be facilitated by providing a stop aid (160; 180; 190).

By using a stop aid (160; 180; 190), the structure (100) is stopped in a defined erected configuration, i.e. with a defined caliper (which, however, is higher than the caliper of the structure in its flat configuration), even if the force along the longitudinal dimension is continued to be applied. The stop aid (160; 180; 190) may ensure that the structure (100) is stopped in the erected configuration with the highest caliper as enabled by the free intermediate portion (137) of the ligaments (130) while the force in the longitudinal dimension is continued to be applied. Alternatively, the stop aid (160; 180; 190) can also facilitate that the structure (100) is stopped in the erected configuration with a certain caliper, which is higher than the caliper of the initial flat configuration but lower than the highest possible caliper which would be possible due to the longitudinal dimension of the free intermediate portion (137) of the ligaments (130). Generally, the stop aid (160; 180: 190), when comprised by the structure (100), can avoid that the structure “over-expands” when a force in the longitudinal dimension is applied, such that the ligaments cannot transition from an initial flat configuration into an erected configuration and further onto a flattened configuration in which the ligaments are turned over by 180°.

There are many different ways to provide a stop aid (160; 180: 190), for example:
a) The first and second layer (110, 120) are attached to each other in at least one layer-on-layer attachment region (160), which may for example be longitudinally outboard of the region where the ligaments (130) are provided, towards one of the lateral edges (114, 124) of the first and/or second layer (110, 120). This layer-on-layer attachment region (160) is provided such that one of the first and second layers (110, 120) has at least one predefined leeway, which may be between two neighboring ligaments, or may alternatively or in addition be between the layer-on-layer attachment region (160) and the ligament attachment region (135, 136) of that ligament which is closest to the layer-on-layer attachment region (160). It is also possible to provide more than one predefined leeway which, in combination, define the maximum possible elongation of the structure. A leeway can form kind of a slack (170) when the structure (100) is in its initial flat configuration, i.e. the longitudinal dimension of the first and/or second layer in the leeway is larger than the longitudinal dimension of the structure in the area where the leeway is provided. An example of such stop aid is shown in FIGS. 1A, 1B, 2, 3 and 6). Alternatively, the leeway can be generated by adapting the material of the first or second layer (110, 120) in the area where the leeway is to be provided to create extensibility of the respective layer in this area. Adapting the material can be done by modifying the material e.g. by selling (weakening the material of the respective first or second layer in the leeway to render it relatively easily extensible), creating holes or using extensible materials to form the leeway. Alternatively, the first or second layer may be made of different material in the area of the leeway, with the material in the leeway being extensible.

It is also possible to provide a leeway that is a combination of a slack and the provision of extensible material in the leeway, such that, upon elongation, initially the slack straightens out and subsequently, the extensible material elongates.

When a force is applied along the longitudinal dimension of the structure (100) to extend the structure, the first and second layers (110, 120) shift against each other in opposite longitudinal directions, the ligaments are erected and the caliper of the structure (100) increases while the length of the structure increases simultaneously. When the first and second layers (110, 120) have been shifted against each other such that the leeway in form of a slack (170), which has been present in the initial flat configuration of the structure, has flattened and straightened out, the structure (100) cannot be extended any further upon application of a force in the longitudinal dimension. Hence, shifting is stopped and the structure has reached its “final” length and caliper in the erected configuration. If the leeway is formed by creation of extensibility in the first or second layer as described above, the first or second layer elongates when the first and second layers (110, 120) are shifted against each other until elongation is not possible any longer (without applying an excessive amount of force, which may even rupture the structure). Hence, the material in the leeway has reached its maximum elongation i.e. it cannot be elongated further upon application of force without causing damage to the structure that limits or impedes its intended use.

Either only one layer-on-layer attachment region (160) can be provided, or, alternatively, two layer-on-layer attachment regions (160) can be provided, one in each of the first and second layer (110, 120). If two layer-on-layer attachment regions (160) are provided, one or more leeway(s) is/are provided in the first layer (110), e.g. towards one of the lateral edges (114) and one or more other leeway(s) is/are provided in the second layer (120), e.g. towards the respective other lateral edge (124) of the structure. Upon extending the structure (100) by applying a force along the longitudinal dimension, the leeway(s) will flatten and straighten out, or if one or more leeway(s) have been obtained by rendering the first or second layer extensible in the respective area, this/these leeway(s) will elongate until they have reached their maximum elongation.

It is also possible to provide a leeway that is a combination of a slack and the provision of extensible material in the leeway, such that, upon elongation, initially the slack straightens out and subsequently, the extensible material elongates.

The material of the layer-on-layer leeway may also be elastic. For such structures, the layer-on-layer stop aid (160) can retract when the force is no longer applied onto the structure such that the structure can substantially “snap back” into its initial flat configuration.

However, if the leeway is extensible (but non-elastic) or if the leeway is elastic, the properties of the leeway have to be such that the leeway does not elongate further when a certain elongation has been reached (i.e. when the structure has erected to the predetermined, desired extend). For many extensible and elastic materials, the materials elongate when a certain force is applied until a certain extension has been reached. Then, due to the material properties, a considerably higher force is needed (often referred to as “force wall”). Thereafter, upon further elongation, the material breaks and ruptures (as may be the case for any other materials when an excessively high force is applied). Selecting appropriate materials and properties for a given application of the structure will be based on the technical knowledge of persons familiar with such materials.

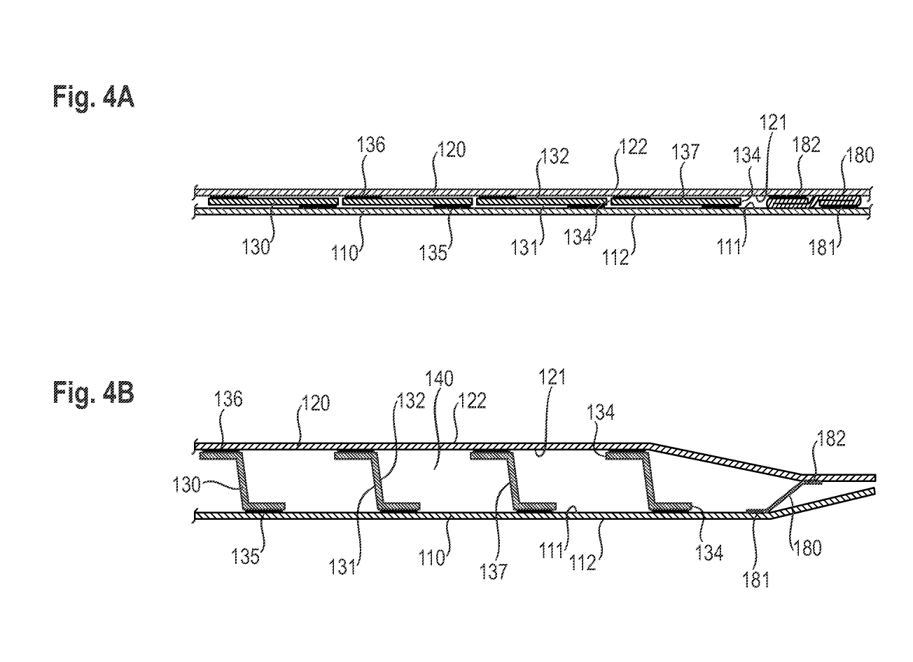

Attachment of the first and second layer (110, 120) to each other in the one or more layer-on-layer attachment regions (160) can be obtained by any means known in the art, such as adhesive, thermal bonding, mechanical bonding (e.g. pressure bonding), ultrasonic bonding, or combinations thereof.
b) A layer-to-layer stop aid (180) may be provided, which extends from the first layer (110) to the second layer (120). This layer-to-layer stop aid (180) is attached to the inner surface (111) or outer surface (112) of the first layer (110) in a first layer-to-layer stop aid attachment region (181) and is further attached to the inner surface (121) or outer surface (122) of the second layer (120) in a second layer-to-layer stop aid attachment region (182). The first layer-to-layer stop aid attachment region (181) may be longitudinally spaced apart from the second layer-to-layer stop aid attachment region (182) when the structure is in its initial flat configuration. The layer-to-layer stop aid (180) is provided with a layer-to-layer stop aid leeway between the first and second layer-to-layer stop aid attachment regions (181, 182) when the structure (100) is in its initial flat configuration. The leeway may be configured in form of a slack (183). Upon application of a force along the longitudinal dimension the first and second layers (110, 120) shift relative to each other in opposite longitudinal directions, the structure extends and erects, and the slack (183) forming the layer-to-layer stop aid leeway between the first and second layer-to-layer stop aid attachment regions (181, 182) straightens out. Once the slack (183) is flattened when the structure (100) is in its erected position, further longitudinal extension of the structure is inhibited also when the force in the longitudinal dimension is continued to be applied. An example of a layer-to-layer stop aid (180) is illustrated in FIGS. 4A (initial flat configuration) and 4B (erected configuration).

Alternatively, the leeway of the layer-to-layer stop aid (180) can be generated by adapting the material between the first and second layer-to-layer stop aid attachment regions (181, 182) to create extensibility of the respective area of the layer-to-layer stop aid (180). Adapting the material can be done by modifying the material, e.g. by selfing (weakening the material of the first or second layer to render it relatively easily extensible), creating holes or using extensible materials to form the leeway. Alternatively, the layer-to-layer stop aid (180) may be made of extensible material. For such layer-to-layer stop aids (180), the material of the leeway elongates when the first and second layers (110, 120) are shifted against each other until elongation of the layer-to-layer stop aid leeway is not possible any longer (without applying an excessive amount of force, which may even rupture the structure). Hence, the material in the leeway has reached its maximum elongation i.e. it cannot be elongated further upon application of force without causing damage to the structure that limits or impedes its intended use.

The material of the leeway may also be elastic. For such structures, the layer-to-layer stop aid (180) can retract when the force is no longer applied onto the structure such that the structure can substantially “snap back” into its initial flat configuration.

For the material properties and appropriate selection of extensible or elastic leeways, the same considerations apply as are set out above for the layer-on-layer stop aid.

It is also possible to provide a leeway that is a combination of a slack and the provision of extensible material in the leeway, such that, upon elongation, initially the slack straightens out and subsequently, the extensible material elongates.

Compared to the attachment of the ligaments to the first and second layer and the resulting configuration, the layer-to-layer stop aid, when applied between the first and second layer and being attached to the inner surfaces of the first and second layer, does not adopt a Z-like or C-like shape having the same orientation as the ligaments with Z-like or C-like shape (otherwise, it would simply be an additional ligament). Instead, the first layer-to-layer stop aid attachment region may be between the first surface of the layer-to-layer stop aid and the inner surface of the first layer. The second layer-to-layer stop aid attachment region may be between the first surface of the layer-to-layer stop aid (i.e. on the same surface of the layer-to-layer stop aid as the first layer-to-layer stop aid attachment region) and the inner surface of the second layer. Thus, the layer-to-layer stop aid adopts a C-like shape when the structure is in its erected configuration.

Alternatively or additionally, the first layer-to-layer stop aid attachment region may be between a first surface of the layer-to-layer stop aid and the inner surface of the first layer. The second layer-to-layer stop aid attachment region may be between the second surface of the layer-to-layer stop aid (i.e. opposite surface of the layer-to-layer stop aid than the first layer-to-layer stop aid attachment region) and the inner surface of the second layer. Thus, the layer-to-layer stop aid adopts a Z-like shape configuration when the structure is in its erected configuration.

Attachment of the layer-to-layer stop aid (180) to the first and second layer (110, 120) in the first and second layer-to-layer stop aid attachment regions (181, 182) can be obtained by any means known in the art, such as adhesive, thermal bonding, mechanical bonding (e.g. pressure bonding), ultrasonic bonding, or combinations thereof.

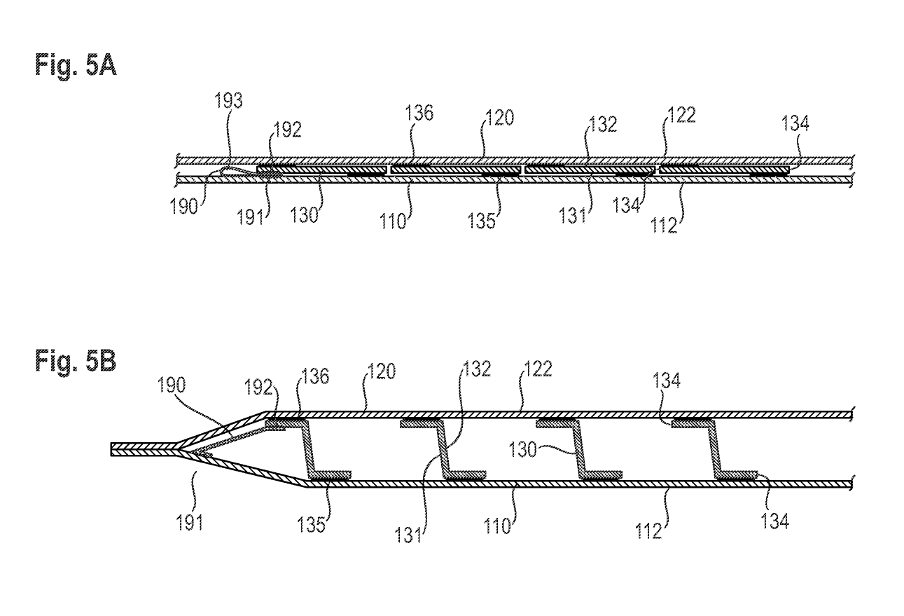

The layer-to-layer stop aid (180) may be provided in combination with another stop aid, such as with the layer-to-ligament stop aid (190) described below. However the layer-to-layer stop aid (180) alone is normally sufficient to define the maximum shifting of the first layer (110) and the second layer (120) relative to each other in opposite longitudinal directions upon application of a force along the longitudinal dimension.
c) A layer-to-ligament stop aid (190) may be provided, which extends from the first or second layer (110, 120) to one of the ligaments (130). This layer-to-ligament stop aid (190) is attached to the inner surface (111) or outer surface (112) of the first or second layer (110, 120) in a first layer-to-ligament stop aid attachment region (191) and is further attached to the first surface (131) or second surface (132) of the ligament (130) in a second layer-to-ligament stop aid attachment region (192). The first layer-to-ligament stop aid attachment region (191) may be longitudinally spaced apart from the second layer-to-ligament stop aid attachment region (192). The layer-to-ligament stop aid (190) is provided with a layer-to-ligament stop aid leeway between the first and second layer-to-ligament stop aid attachment region (191, 192) when the structure (100) is in its initial flat configuration. The leeway may be configured in form of a slack (193). Upon application of a force in the longitudinal dimension the first and second layer (110, 120) shift relative to each other in opposite longitudinal directions, the structure extends and erects, and the slack (193) forming the layer-to-ligament stop aid leeway between the first and second layer-to-ligament stop aid attachment regions (191, 192) straightens out. Once the slack (193) is straightened out when the structure (100) is in its erected position, further longitudinal extension of the structure is inhibited also when the force in the longitudinal dimension is continued to be applied. A layer-to-ligament stop aid (190) is shown in FIGS. 5A (initial flat configuration) and 5B (erected configuration).

Alternatively or additionally, the leeway of the layer-to-ligament stop aid (190) can be generated by adapting the material between the first and second layer-to-ligament stop aid attachment regions (191, 192) to create extensibility of the respective area of the layer-to-ligament stop aid (190). Adapting the material can be done by modifying the material, e.g. by selling (weakening the material of the first or second layer to render it relatively easily extensible), creating holes or using extensible materials to form the leeway.

Alternatively or additionally, the layer-to-ligament stop aid (190) may be made of extensible material. For such layer-to-ligament stop aids (190), the material of the leeway elongates when the first and second layers (110, 120) are shifted against each other until elongation of the layer-to-ligament stop aid leeway is not possible any longer (without applying an excessive amount of force, which may even rupture the structure). Hence, the material in the leeway has reached its maximum elongation i.e. it cannot be elongated further upon application of force without causing damage to the structure that limits or impedes its intended use.

The material of the leeway may also be elastic. For such structures, the layer-to-ligament stop aid (190) can retract when the force is no longer applied onto the structure such that the structure can substantially “snap back” into its initial flat configuration.

For the material properties and appropriate selection of extensible or elastic leeways, the same considerations apply as are set out above for the layer-on-layer stop aid.

It is also possible to provide a leeway that is a combination of a slack and the provision of extensible material in the leeway, such that, upon elongation, initially the slack straightens out and subsequently, the extensible material elongates.

Attachment of the layer-to-ligament stop aid (190) to the first and second layer (110, 120) in the first and second layer-to-layer stop aid attachment regions (181, 182) can be obtained by any means known in the art, such as adhesive, thermal bonding, mechanical bonding (e.g. pressure bonding), ultrasonic bonding, or combinations thereof.

The layer-to-ligament stop aid (190) may be provided in combination with another stop aid, such as with the layer-to-ligament stop aid (190) or with the layer-on-layer stop aid as are described below. However the layer-to-ligament stop aid (190) alone is normally sufficient to define the maximum shifting of the first layer (110) and the second layer (120) relative to each other in opposite longitudinal directions upon application of a force along the longitudinal dimension. Alternatively, the leeway of the layer-to-ligament stop aid (190) can be generated by adapting the material between the first and second layer-to-ligament stop aid attachment regions (191, 192) to create extensibility of the respective area of the layer-to-ligament stop aid (190). Adapting the material can be done by modifying the material, e.g. by selling (weakening the material of the first or second layer to render it relatively easily extensible), creating holes or using extensible materials to form the leeway. Alternatively, the layer-to-ligament stop aid (180) may be made of extensible material. For such layer-to-ligament stop aids (180), the material of the leeway elongates when the first and second layers (110, 120) are shifted against each other until elongation of the layer-to-ligament stop aid leeway is not possible any longer (without applying an excessive amount of force, which may even rupture the structure). Hence, the material in the leeway has reached its maximum elongation i.e. it cannot be elongated further upon application of force without causing damage to the structure that limits or impedes its intended use. The material of the leeway may also be elastic. For such structures, the layer-to-ligament stop aid (190) can retract when the force is no longer applied onto the structure such that the structure can substantially “snap back” into its initial flat configuration.

Generally, the layer-to-ligament stop aid (190) may be attached in the first and second layer-to-ligament stop aid attachment regions such that the layer-to-ligament stop aid extends along or adjacent to one of the longitudinal edges of the at least one ligament (130) or, alternatively, such that it extends between the longitudinal edges of the at least one ligament.

Attachment of the layer-to-ligament stop aid (190) to the first or second layer (110, 120) and the ligament (130) in the first and second layer-to-ligament stop aid attachment regions (191, 192) can be obtained by any means known in the art, such as adhesive, thermal bonding, mechanical bonding (e.g. pressure bonding), ultrasonic bonding, or combinations thereof.

The layer-to-ligament stop aid (190) may be provided in combination with another stop aid, such as with a layer-to-layer stop aid (180). However, the layer-to-ligament stop aid (190) alone is normally sufficient to define the maximum shifting of the first layer (110) and the second layer (120) relative to each other in opposite longitudinal directions upon application of a force along the longitudinal dimension.
d) The structure (100) may comprise an enveloping stop aid (not shown) which encircles at least a portion of the first and second layer (110, 120) and the ligaments (130) provided between the first and second layer in the respective portion. This enveloping stop aid is attached to the first layer (110), the second layer (120) and/or at least one of the ligaments (130) in one or more enveloping stop aid attachment region. Attaching the enveloping stop aid to only one of the first layer (110), the second layer (120) or at least one of the ligaments (130) in only one enveloping stop aid attachment region is, however, sufficient.

The enveloping stop aid is attached to itself to form a closed loop with a defined circumference around at least a portion of the first and second layer with the ligaments in between. The enveloping stop aid may encircle the first and second layer (110, 120) along the longitudinal dimension or along the lateral dimension. Generally, if the enveloping stop aid encircles the first and second layer (110, 120) along the lateral dimension, the risk of the enveloping stop aid sliding off the first and second layer (110, 120) upon elongation and erection of the structure may be lower compared to the enveloping stop aid encircling the first and second layer along the longitudinal dimension, especially for rather long structures. However, by providing further enveloping stop aid attachment regions, such risk can be reduced.

The circumference of the enveloping stop aid defines the maximum shifting of the first layer (110) and the second layer (120) relative to each other in opposite longitudinal directions upon application of a force along the longitudinal dimension.

When the structure (100) is in its initial flat configuration, the enveloping stop aid is loose around the first and second layer (and the respective ligaments between the first and second layer). Upon application of a force along the longitudinal dimension of the structure, the structure erects until the enveloping stop aid fits tightly around the first and second layer (and the respective ligaments between the first and second layer), which will stop further shifting of the first layer relative to the second layer also if the force along the longitudinal dimension is continued to be applied. To assist in avoiding overexpansion of the structure (100), the circumference of the enveloping stop aid may be such that further shifting of the first layer (110) relative to the second layer (120) along the longitudinal dimensions is inhibited before the ligaments (130) are in their fullest upright position.

General considerations for the layer-to-layer stop aid, the layer-to-ligament stop aid and, if expressly mentioned, the enveloping stop aid:

The layer-to-layer stop aid and/or the layer-to-ligament stop aid may be non-elastic or highly non-elastic (apart from the leeway, if the leeway is provided by modifying the material to render it elastically extensible). Also the layer-to-layer stop aid and/or the layer-to-ligament stop aid may be non-extensible or highly non-extensible (apart from the leeway, if the leeway is provided by modifying the material to render it extensible).

The layer-to-layer stop aid and/or the layer-to-ligament stop aid and/or enveloping stop aid can be made of a sheet-like material, such as nonwoven, film, paper, sheet-like foam, woven fabric, knitted fabric, or combinations of these materials. Combinations of these materials may be laminates, e.g. a laminate of a film and a nonwoven. The layer-to-layer stop aid and/or the layer-to-ligament stop aid and/or enveloping stop aid may also be made of a cord- or string-like material.

The layer-to-layer stop aid and/or the layer-to-ligament stop aid and/or enveloping stop aid is not necessarily intended to contribute to the resistance of the structure against a force exerted onto the structure in the thickness-direction. However, the basis weight, tensile strength and bending stiffness of the layer-to-layer stop aid and/or the layer-to-ligament stop aid and/or enveloping stop aid should be sufficiently high to avoid inadvertent tearing of the layer-to-layer stop aid and/or the layer-to-ligament stop aid and/or enveloping stop aid upon expansion of the structure.

If the layer-to-layer stop aid and/or the layer-to-ligament stop aid and/or enveloping stop aid is made of a sheet-like material, the basis weight of the layer-to-layer stop aid and/or the layer-to-ligament stop aid and/or enveloping stop aid may be at least 1 g/m², or at least 2 g/m², or at least 3 g/m², or at least 5 g/m²; and the basis weight may further be not more than 500 g/m², or not more than 200 g/m², or not more than 100 g/m², or not more than 50 g/m², or not more than 30 g/m².

If the layer-to-layer stop aid and/or the layer-to-ligament stop aid and/or enveloping stop aid is made of a cord- or string-like material, the basis weight of the layer-to-layer stop aid and/or the layer-to-ligament stop aid and/or enveloping stop aid may be at least 1 gram per meter (g/m), or at least 2 g/m, or at least 3 g/m, or at least 5 g/m; and the basis weight may further be not more than 500 g/m, or not more than 200 g/m, or not more than 100 g/m, or not more than 50 g/m, or not more than 30 g/m.

The basis weight of the layer-to-layer stop aid and/or the layer-to-ligament stop aid may be less than the basis weight of the ligaments, for example the basis weight of the layer-to-layer stop aid and/or the layer-to-ligament stop aid may be less than 80%, or less than 50% of the basis of the ligaments (if the ligaments vary in basis weight, then these values are with respect to the iigament(s) with the lowest basis weight).

The tensile strength of the layer-to-layer stop aid may be at least 2 N/cm, or at least 4 N/cm or at least 5 N/cm. The tensile strength may be less than 100 N/cm, or less than 80 N/cm, or less than 50 N/cm, or less than 30 N/cm, or less than 20 N/cm.

The bending stiffness of the layer-to-layer stop aid and/or the layer-to-ligament stop aid and/or enveloping stop aid may be at least 0.1 mNm, or at least 0.2 mNm, or at least 0.3 mNm. The bending stiffness may be less than 200 mNm, or less than 150 mNm, or less than 100 mNm, or less than 50 mNm, or less than 10 mNm, or less than 5 mNm. These values apply to sheet-like layer-to-layer stop aids and/or layer-to-ligament stop aids and/or enveloping stop aids, for cord- or string-like layer-to-layer stop aids and/or layer-to-ligament stop aids and/or enveloping stop aids, the bending stiffness is generally not seen as critical.

The tensile strength of the layer-to-layer stop aid and/or the layer-to-ligament stop aid and/or enveloping stop aid may be lower than the tensile strength of the ligaments, for example the tensile strength of the layer-to-layer stop aid may be less than 80%, or less than 50% of the tensile strength of the ligaments (if the ligaments vary in tensile strength, then these values are with respect to the ligament(s) with the lowest tensile strength).

The bending stiffness of the layer-to-layer stop aid and/or the layer-to-ligament stop aid and/or enveloping stop aid (when made of sheet-like material) may be lower than the bending stiffness of the ligaments, for example the bending stiffness of the layer-to-layer stop aid and/or the layer-to-ligament stop aid and/or enveloping stop aid may be less than 80%, or less than 50% of the bending stiffness of the ligaments (if the ligaments vary in bending stiffness, then these values are with respect to the ligament(s) with the lowest bending stiffness).

Alternatively or in addition to the provision of a stop aid comprised by the structure, the maximum possible elongation and erection of the structure can also be determined by a means that is comprised by the (disposable) consumer product, which comprises the structure. Such means does not need to be in direct contact with the structure.

### Use of the Structures

The structures of the present invention can be used in a large variety of consumer products. Examples are wound dressings or bandages. Wound dressings and bandages comprising one or more structures of the present invention, can be held in intimate contact with parts of a human or animal body are with a wound. In addition, the structures of the present invention can provide a buffering effect, acting as antishocks in case the part of a body or wound which are covered by the bandage or wound dressing is unintentionally bounced against a hard surface, due to the ability of the structure to increase in caliper when being elongated.

If one or more structures of the present invention are comprised by a flexible packaging (wherein the flexible packaging may be made of film), the structures can provide a cushioning effect, thus assisting in protecting the contents of the flexible package. Furthermore, a flexible packaging comprising one or more structures of the present invention can fill areas within the packaging which would otherwise be empty due to the shape of the products contained in the packaging. Thereby, the structures can help to balance or avoid packaging deformations. This can allow for e.g. more rectangular packaging shape, which enables easier handling and storage (especially when several packages are stacked upon each other).

### Test Methods:

### Tensile Strength

Tensile Strength is measured on a constant rate with extension tensile tester Zwick Roell Z2.5 with computer interface, using TestExpert 11.0 Software, as available from Zwick Roell GmbH &Co. KG, Ulm, Germany. A load cell is used for which the forces measured are within 10% to 90% of the limit of the cell. Both the movable (upper) and stationary (lower) pneumatic jaws are fitted with rubber faced grips, wider than the width of the test specimen. All testing is performed in a conditioned room maintained at about 23° C.+2° C. and about 45%±5% relative humidity.

With a die or razor knife, cut a material specimen which is 25.4 mm wide and 100 mm long. For the present invention, the length of the specimen correlates to the longitudinal dimension of the material within the structure.

If the ligament is smaller than the size of the material specimen specified in the previous paragraph, the material specimen may be cut from a larger piece such as the raw material used for making the ligaments. Care should be taken to correlate the orientation of such specimen accordingly, i.e. with the length of the specimen correlating to the longitudinal dimension of the material within the structure. However, if the ligament has a width somewhat smaller than 25.4 mm (e.g. 20 mm, or 15 mm) the width of the specimen can be accordingly smaller without significantly impacting the measured tensile strength.

If the ligament comprises different materials in different regions, the tensile strength of each material can be determined separately by taking the respective raw materials. It is also possible to measure the tensile strength of the overall ligament. However, in this case, the measured tensile test will be determined by the material within the ligament which has the lowest tensile strength.

Precondition the specimens at about 23° C.±2° C. and about 45%±5% relative humidity for 2 hours prior to testing.

For analyses, set the gauge length to 50 mm. Zero the crosshead and load cell. Insert the specimen into the upper grips, aligning it vertically within the upper and lower jaws and close the upper grips. Insert the specimen into the lower grips and close. The specimen should be under enough tension to eliminate any slack, but less than 0.025 N of force on the load cell.

Program the tensile tester to perform an extension test, collecting force and extension data at an acquisition rate of 50 Hz as the crosshead raises at a rate of 100 mm/min until the specimen breaks. Start the tensile tester and data collection. Program the software to record Peak Force (N) from the constructed force (N) verses extension (mm) curve. Calculate tensile strength as:

Tensile Strength=Peak Force (N)/width of specimen (cm)

For rope/string like materials: tensile strength−peak force (N)

Analyze all tensile Specimens. Record Tensile Strength to the nearest 1 N/cm. A total of five test samples are analyzed in like fashion. Calculate and report the average and standard deviation of Tensile Strength to the nearest 1 N/cm for all 5 measured specimens.

### Bending Stiffness

Bending stiffness is measured using a Lorentzen & Wettre Bending Resistance Tester (BRT) Model SE016 instrument commercially available from Lorentzen & Wettre GmbH, Darmstadt, Germany. Stiffness off the materials (e.g. ligaments and first and second layer) is measured in accordance with SCAN-P 29:69 and corresponding to the requirements according to DIN 53121 (3.1 “Two-point Method”). For analysis a 25.4 mm by 50 mm rectangular specimen was used instead of the 38.1 mm by 50 mm specimen recited in the standard. Therefore, the bending force was specified in mN and the bending resistance was measured according to the formula present below.

The bending stiffness is calculated as follows:

$${S}_{b} = \frac{60 \times F \times {l}^{2}}{π \times α \times b}$$

with:
S_{b}=bending stiffness in mNm
F=bending force in N
l=bending length in mm
α=bending angle in degrees
b=sample width in mm

With a die or razor knife, cut a specimen of 25.4 mm by 50 mm whereby the longer portion of the specimen corresponds to the lateral dimension of the material when incorporated into a structure. If the materials are relatively soft, the bending length “l” should be 1 mm. However, if the materials are stiffer such that the load cell capacity is not sufficient any longer for the measurement and indicates “Error”, the bending length “l” has to be set at 10 mm. If with a bending length “l” of 10 mm, the load cell again indicates “Error”, the bending length “l” may be chosen to be more than 10 mm, such as 20 mm or 30 mm. Alternatively (or in addition, if needed), the bending angle may be reduced from 30° to 10°.

For the ligament data given in Table 1, the bending length “l” was 10 mm. For the 1^{st} and 2^{nd} layer materials the bending length “l” was 1 mm. The bending angel has been 30° for the ligaments as well as for the 1^{st} and 2^{nd} layer.

Precondition the specimen at about 23° C.±2° C. and about 45%±5% relative humidity for two hours prior to testing.

Regarding the size of the ligament and the procedure in case the size of the ligament is smaller than the size of the specimen, the same applies as set out above for the tensile test.

TABLE 1
Basis weight, tensile strength and bending stiffness of suitable first
and second layers materials:

| | Example No. | | | | | | | |
|---|---|---|---|---|---|---|---|---|
| | 1 | | 2 | | 3 | | 4 | |
| Basis weight of | 13 | g/m² | 15 | g/m² | 17 | g/m² | 25 | g/m² |
| 1^{st} and 2^{nd} layer | | | | | | | | |
| Bending stiffness of 1^{st} | 0.3 | mNm | 0.4 | mNm | 0.5 | mNm | 0.9 | mNm |
| and 2^{nd} layer | | | | | | | | |
| Tensile strength of | 6.9 | N/cm | 7.9 | N/cm | 7.8 | N/cm | 8.1 | N/cm |
| 1^{st} and 2^{nd} layer | | | | | | | | |

Example materials 1 to 4 are all spunbond polypropylene nonwovens.

TABLE 2
Basis weight, tensile strength and bending stiffness of suitable
ligament materials:

| | Example No. | | | | | | | |
|---|---|---|---|---|---|---|---|---|
| | 1 | | 2 | | 3 | | 4 | |
| Basis weight | 40 | g/m² | 43 | g/m² | 51 | g/m² | 60 | g/m² |
| of ligaments | | | | | | | | |
| Bending | 10.8 | mNm | 36.3 | mNm | 52.6 | mNm | 105.3 | mNm |
| stiffness of | | | | | | | | |
| ligaments | | | | | | | | |
| Tensile | 16.2 | N/cm | 16.1 | N/cm | 18.8 | N/cm | 26.1 | N/cm |
| strength of | | | | | | | | |
| ligaments | | | | | | | | |

Except for Example 1 (40 g/m² material), all ligament materials were spunbond PET nonwovens. Example 1 was a spunbond polypropylene material.

### Method to Measure Ligament Caliper

Average Measured caliper is measured using a Mitutoyo Absolute caliper device model ID-C1506, Mitutoyo Corp., Japan.

A sample of the material used for the ligaments with a sample size of 40 mm×40 mm is cut. If the samples are taken from a ready-made structure and the size of the ligaments is smaller than 40 mm×40 mm, the sample may be assembled by placing two or more ligaments next to each other with no gap and no overlap between them. Precondition the specimens at about 23° C.±2° C. and about 45%±5% relative humidity for 2 hours prior to testing.

Place the measuring plate on the base blade of the apparatus. Zero the scale when the probe touches the measuring plate (Measuring plate 40 mm diameters, 1.5 mm height and weight of 2.149 g). Place the test piece on the base plate. Place the measurement plate centrally on top of the sample without applying pressure. After 10 sec. move the measuring bar downwards until the probe touches the surface of the measuring plate and read the caliper from the scale to the nearest 0.01 mm.

### Method of Measuring Modulus of the Structure

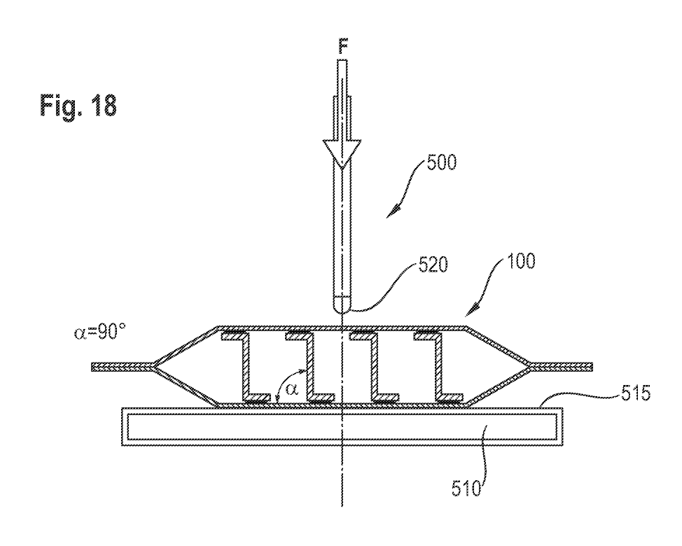

The modulus of the structure is measured on a constant rate of structure compression using a tensile tester with computer interface (a suitable instrument is the Zwick Roell Z2.5 using Test-Expert 11.0 Software, as available from Zwick Roell GmbH &Co. KG, Ulm, Germany) using a load cell for which the forces measured are within 10% to 90% of the limit of the cell. The movable upper stationary pneumatic jaw is fitted with rubber faced grip to securely clamp the plunger plate (500). The stationary lower jaw is a base plate (510) with dimensions of 100 mm×100 mm. The surface of the base plate (510) is perpendicular to the plunger plate (500). To fix the plunger plate (500) to the upper jaw, lower the upper jaw down to 20 mm above the upper surface (515) of the base plate (510). Close the upper jaw and make sure the plunger plate (500) is securely tightened. Plunger plate (500) has a width of 3.2 mm and a length of 100 mm. The edge (520) of the plunger plate (500) which will contact the structure has a curved surface with an impacting edge radius of r=1.6 mm. For analysis, set the gauge length to at least 10% higher than the caliper of the structure in its erected configuration (see FIG. 18). Zero the crosshead and load cell. The width of the plunger plate (500) should be parallel with the transverse direction of the structure.

Precondition samples at about 23° C.±2° C. and about 45% RH±5% RH relative humidity for 2 hours prior to testing. The structure is placed on the base plate, is transformed into its erected configuration and fixed in its erected configuration with substantially maximum possible caliper to the base plate with the outer surface of its first (lower) layer facing towards the upper surface (515) of the base plate (510). The structure can be fixed to the upper surface of the base plate, e.g. by placing adhesive tapes on the lateral edges of the structure's first (lower) layer and fix the tapes to the upper surface of the base plate.

Program the tensile tester to perform a compression test, collecting force and travel distance data at an acquisition rate of 50 Hz as the crosshead descends at a rate of 50 mm/min from starting position to 2 mm above base plate (safety margin to avoid destruction of load cell).

If the modulus of the structure is measured directly in an area where a ligament is placed, the force P [N] is the force when the indentation depth h [mm] of the plunger plate into the structure is equal to 50% of the longitudinal dimension of the free intermediate portion of the ligament below the plunger plate.

If the modulus of the structure is measured between two neighboring ligaments, the force P [N] is the force when the indentation depth h [mm] of the plunger plate into the structure is equal to 50% of the longitudinal dimension of the free intermediate portion of the two ligaments nearest to the plunger plate (i.e. the ligaments on each side of the plunger plate as seen along the longitudinal structure dimension). If the free intermediate portion of the two neighboring ligaments, between which the modulus is measured, differ from each other with respect to the longitudinal dimension of their free intermediate portions, the average value over these two free intermediate portions is calculated and the indentation depth h [mm] of the plunger plate into the structure is equal to 50% of this average free longitudinal dimension.

A total of three test specimens are analyzed in like fashion.

The modulus E [N/mm²] is calculated as follows:

$$E = \frac{3 P}{8 rh} .$$

With r being the impacting edge radius of the plunger plate, i.e. r=1.6 mm

Calculate and report the average of modulus E for all 3 measured specimens.

All testing is performed in a conditioned room maintained at about 23° C.+2° C. and about 45% RH±15% relative humidity.

### Example Structures

### Making of Example Structures:

For each structure, cut 2 pieces of nonwovens with longitudinal dimension of 150 mm and a lateral dimension of 25 mm, with a die or razor knife. These nonwovens will become the first and second layers of the structure. Cut 4 ligaments (Examples 1, 2, 4 and 5), respectively 6 ligaments (Example 3) with a longitudinal dimension of 10 mm (which will result in a longitudinal dimension of the free intermediate portion of 4 mm in the final structures, while 3 mm on each lateral edge are attached to the first and second layer, respectively) and a lateral dimension of 25 mm, with a die or razor knife. Apply a double sided tape (e.g. 3M Double sided medical tape 1524-3M (44 g/m²) available from 3M) with length of 3 mm and width of 25 mm on the first surface of the each ligament adjacent the side edge, which will become the first lateral edge of the ligament in the final structure and a second tape on the second surface of each ligament adjacent the side edge, which will become the second lateral edge of the ligament in the final structure. The width of the tape is aligned with the lateral dimension of the ligaments.

Remove one release tape from the ligaments and attach the tape to the first layer the first layer such that the lateral dimension of the ligament is aligned with the lateral dimension of the first layer.

For Example 1: Place the first, second, third and fourth ligaments such that the spacing between neighboring ligaments is 10 mm when the ligaments are lying flat on the first layer.

For Example 2, 4 and 5: Place the first, second, third and fourth ligaments such that the spacing between neighboring ligaments is 5 mm when the ligaments are lying flat on the first layer.

For Example 3: In this example, 6 ligaments where positioned between the first and second layer. Place the first to sixth ligaments such that there is no spacing between neighboring ligaments and neighboring ligaments overlap by 3 mm with each other when the ligaments are lying flat on the first layer.

For all examples, the ligaments should be positioned accordingly, to leave sufficient space at the lateral edges of the first and second layer to allow attaching the first layer to the second layer in the manner described below.

For all Examples structures, the 1^{st} and 2^{nd} layer were made of the material of Example 2 of Table 1. The ligaments for the Example structures 1, 2, 3 and 5 were made of the ligament material of Example 4 of Table 2, while the ligaments of Example structure 4 were made of the ligament material of Example 1 of Table 2.

Remove the remaining release layers from the remaining tape pieces on all four ligaments and attach the second layer on top of the first layer and the ligaments such that the second layer is congruent with the first layer.

To bond the first layer to the second layer in the areas longitudinally outwardly from the area where the ligaments are placed (stop aid), two double-sided tapes (e.g. 3M Double sided medical tape 1524-3M (44 g/m²) available from 3M) having a length of 3 mm and a width of 25 mm are provided. A first tape is attached to the first layer towards one of the first layer's lateral edges such that the distance between this first tape and the adjacent ligament is 20 mm. The second tape is attached to the first layer towards the respective other lateral edge of the first layer such that the distance between this second tape and the respective adjacent ligament is 20 mm. The width of the first and second tape is aligned with the lateral dimension of the first layer. Pay attention that the first and second tapes are not attached to the second layer before the structure has been transformed into its erected configuration (see next step).

Stretch the resulting cell forming structure along the longitudinal dimension into the erected configuration such that the first and second layer shift in opposite directions and the ligaments move in upright position of 90° relative to the first and second layer. Notably, the 90° does not apply to the area longitudinally outwardly from the outermost ligaments (viewed along the longitudinal dimension) because the first and second layers follow a tapered path in this area until the point where they coincide with each other (see e.g. FIG. 1B). Maintain the structure in its erected configuration and attach the first layer to the second layer via the first and second tape to fix the structure in its erected configuration. Release the force and allow the structure to relax,

For the Structure Modulus Test, place the assembled cell forming structure flat and un-stretched under the plunger plate. Stretch the cell forming structure such that in the erected configuration the ligaments are approximately at an angle of 90° versus the first and second layer in the areas between neighboring ligaments.

The plunger plate was placed centrally in the space between the two center ligaments of the structure, except for Example 5, where the plunger plate was placed centrally directly in the location where one of the two centrally positioned (viewed along the longitudinal direction of the structure) ligaments is. The erected structure was fixed to the base plate using tape. The test procedure set out above for the structure modulus was followed.

TABLE 3
Modulus of the structure

| | Example Structure No. | | | | |
|---|---|---|---|---|---|
| | 1 | 2 | 3 | 4 | 5 |
| Modulus | 0.028 | 0.039 | 0.107 | 0.005 | 0.151 |
| [N/mm²] | | | | | |

The data show that the measured modulus of a structure will be different depending e.g. on whether the modulus is measured between neighboring ligaments (Examples 1 to 4), or if it is measured directly in the location where a ligament is attached to the first and second layer. For a given structure, between neighboring ligaments, the modulus will typically be lower than directly at a ligament.

All patents and patent applications (including any patents which issue thereon) assigned to the Procter & Gamble Company referred to herein are hereby incorporated by reference to the extent that it is consistent herewith.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A structure having a longitudinal dimension and a lateral dimension perpendicular to the longitudinal dimension, and being able to elongate along the longitudinal dimension upon application of a force along the longitudinal dimension of the structure, whereby the structure is simultaneously able to convert from an initial flat configuration into an erected configuration,
wherein the structure comprises a first and a second layer, each layer having an inner and an outer surface, a longitudinal dimension parallel to the longitudinal dimension of the structure confined by two spaced apart lateral edges, and a lateral dimension parallel to the lateral dimension of the structure confined by two spaced apart longitudinal edges, wherein the first and second layer at least partly overlap each other, wherein the first layer is able to shift relative to the second layer in opposite directions along the longitudinal structure dimension upon application of a force along the longitudinal dimension,
the structure further comprising ligaments provided between the first and second layer in at least a part of the region where the first and second layer overlap each other,
wherein the first and second layers and the ligaments are made of film, nonwoven material, paper, sheet-like foam, woven fabric, knitted fabric or combinations thereof,
each ligament has a longitudinal dimension confined by two spaced apart lateral ligament edges, and a lateral dimension confined by two spaced apart longitudinal ligament edges,
each ligament being attached to the inner surface of the first layer with a portion at or adjacent to one of the ligament's lateral edges in a first ligament attachment region and being attached to the inner surface of the second layer with a portion at or adjacent to the ligament's other lateral edge in a second ligament attachment region, the region of each ligament between the first and second ligament attachment region forming a free intermediate portion,
the ligaments being spaced apart from one another along the longitudinal dimension of the structure, and the attachment of all ligaments to the first and second layer in the first and second ligament attachment regions is such that the free intermediate portions of the ligaments are able to convert from an initial flat configuration to an erected configuration upon application of a force along the longitudinal dimension of the structure, thus converting the structure as a whole from an initial flat configuration into an erected configuration, wherein the erection is in the direction perpendicular to the longitudinal and the lateral dimension of the structure.

2. The structure of claim 1, wherein the ligaments, in their erected configuration, either adopt a Z-like shape, a C-like shape, a T-like shape, or a Double-T-like shape.

3. The structure of claim 1, wherein the structure comprises one or more stop aid(s) which define(s) the maximum shifting of the first layer relative to the second layer along the longitudinal dimension in opposite directions when the force along the longitudinal dimension is continued to be applied, wherein the maximum shifting defined by the stop aid is less than the maximum shifting provided by the ligaments in the absence of such stop aid.

4. The structure of claim 3, wherein the one or more stop aid is selected from the group consisting of:
a) the first and second layer being attached to each other in at least one attachment region, wherein the at least one layer-on-layer attachment region is provided such that one of the first and second layer has one or more leeway(s) when the structure is in its initial flat configuration, the leeway preferably being provided between the layer-on-layer attachment region and the ligament which is closest to the layer-on-layer attachment region; said leeway being able to straighten out and/or extend when the structure is transferred into its erected configuration; and
b) a layer-to-layer stop aid extending from the first layer to the second layer and being attached to the first layer in a first layer-to-layer stop aid attachment region and being further attached to the second layer in a second layer-to-layer stop aid attachment region, wherein the layer-to-layer stop aid is provided with a layer-to-layer stop aid leeway between the first layer-to-layer stop aid attachment region and the second layer-to-layer stop aid attachment region when the structure is in its initial flat configuration, said leeway being able to straighten out and/or extend when the structure is transferred into its erected configuration; and
c) a layer-to-ligament stop aid extending from the first or second layer to one of the ligaments and being attached to the first or second layer in a first layer-to-ligament stop aid attachment region and being attached to the ligament in a second layer-to-ligament stop aid attachment region, wherein the layer-to-ligament stop aid is provided with a layer-to-ligament stop aid leeway between the first layer-to-ligament stop aid attachment region and the second layer-to-ligament stop aid attachment region when the structure is in its initial flat configuration, said leeway being able to straighten out and/or extend when the structure is transferred into its erected configuration; and
d) an enveloping stop aid encircling a least a portion of the first and second layer and of the ligaments between the first and second layer, wherein the enveloping stop aid is attached to the first layer, the second layer and/or one or more ligaments in at least one enveloping stop aid attachment region and wherein the enveloping stop aid is further attached to itself to form a closed loop with a defined circumference around a least a portion of the first and second layer and of the ligaments between the first and second layer, wherein the circumference of the enveloping stop aid defines the maximum caliper of the structure in its erected configuration; wherein the caliper is perpendicular to the lateral and longitudinal dimension of the structure; and
e) any combinations of a) to d).

5. The structure of claim 1, wherein the first and second layers are non-elastic, preferably non elastic and non-extensible at least outside the areas where a leeway is provided, which is comprised by a stop aid.

6. The structure of claim 1, wherein the areas where the ligaments are located and the areas between neighboring ligaments have a modulus of from about 0.01 N/mm² to about 0.3 N/mm².

7. The structure of claim 1, wherein the longitudinal dimension of the ligaments in the initial flat configuration of structure is substantially parallel with the longitudinal dimension of the first and second layer.

8. The structure of claim 1, wherein the erected structure configuration, upon release of the force applied along the longitudinal dimension, returns substantially completely to its initial flat configuration.

9. The structure of claim 1, wherein the ligaments differ from each other in tensile strength, in bending stiffness or in tensile strength and bending stiffness.

10. The structure of claim 1, wherein one or more ligaments provided towards the longitudinal center of the structure have a higher tensile strength and/or a higher bending stiffness than the ligaments provided towards the lateral edges of the structure.

11. The structure of claim 1, wherein the lateral dimension of at least some of the ligaments is smaller than the lateral dimension of the first and second layer, wherein the ligaments having a smaller lateral dimension than the lateral dimension of the first and second layer are flanked by laterally neighboring ligaments.

12. The structure of claim 1, wherein in one or more ligaments, those portions of the free intermediate portion which are directly adjacent to the first and/or second ligament attachment region have a different bending stiffness, different tensile strength, or different bending stiffness and tensile strength than the remaining free intermediate portion of said ligament.

13. The structure of claim 1, wherein the first layer and the second layer are made of a single continuous material which is folded over at one of the lateral edges of the structure, such that one of the lateral edges of the first layer is coincident with one of the lateral edges of the second layer, with these lateral edges being located at the interface of the first and second layer.

14. A disposable consumer product comprising at least one structure of claim 1.

15. The disposable consumer product of claim 14, wherein the disposable consumer product is a wound dressing or bandage.

16. A flexible packaging comprising at least one structure of claim 1.
